SEQUENCE LISTING (114 sequences)

SEQ ID NO: 1 - protein, 107 aa, Artificial sequence [sequence omitted]

SEQ ID NO: 2 - protein, 120 aa, Artificial sequence [sequence omitted]

SEQ ID NO: 3 - protein, 15 aa, Artificial sequence
  Synthetic
GGGGSGGGGSGGGGS

SEQ ID NO: 4 - protein, 242 aa, Artificial sequence
  Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 5 - protein, 24 aa, Mycobacterium tuberculosis
MKRGLTVAVAGAAILVAGLSGCSS

SEQ ID NO: 6 - protein, 12 aa, Artificial sequence
  Synthetic
TRHRQPRGWEQL

SEQ ID NO: 7 - protein, 10 aa, Artificial sequence
  Synthetic
GGGGSGGGGS

SEQ ID NO: 8 - protein, 9 aa, Artificial sequence
  Synthetic
GGSGGSGGS

SEQ ID NO: 9 - protein, 21 aa, Artificial sequence
  Synthetic
MKVKVLSLLVPALLVAGAANA

SEQ ID NO: 10 - protein, 6 aa, Artificial sequence
  Synthetic
HHHHHH

SEQ ID NO: 11 - protein, 9 aa, Artificial sequence
  Synthetic
NLVPMVATV

SEQ ID NO: 12 - protein, 342 aa, Artificial sequence
  Synthetic
MKVKVLSLLVPALLVAGAANADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSTRHRQPRGWEQLGGSGGSGGSMKRGLTVAVAGAAILVAGLSGCSSGGSGGSGGSNLVPMVATVHHHHHH

SEQ ID NO: 13 - protein, 336 aa, Artificial sequence
  Synthetic
MKVKVLSLLVPALLVAGAANADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSTRHRQPRGWEQLGGSGGSGGSMKRGLTVAVAGAAILVAGLSGCSSGGSGGSGGSNLVPMVATV

SEQ ID NO: 14 - protein, 321 aa, Artificial sequence
  Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSTRHRQPRGWEQLGGSGGSGGSMKRGLTVAVAGAAILVAGLSGCSSGGSGGSGGSNLVPMVATVHHHHHH

SEQ ID NO: 15 - protein, 315 aa, Artificial sequence
  Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSTRHRQPRGWEQLGGSGGSGGSMKRGLTVAVAGAAILVAGLSGCSSGGSGGSGGSNLVPMVATV

SEQ ID NO: 16 - protein, 297 aa, Artificial sequence
  Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSTRHRQPRGWEQLGGSGGSGGSMKRGLTVAVAGAAILVAGLSGCSS

SEQ ID NO: 17 - DNA, 1026 bp, Artificial sequence
  Synthetic
atgaaggtgaaagtgctgagcctgctggttccggcgctgctggttgcgggtgcggcgaatgcggacatccaaatgacccaaagcccgagcagcctgagcgcgagcgtgggtgaccgtgttaccatcacctgccgtgcgagccaggatgtgaacaccgcggttgcgtggtatcagcaaaagccgggcaaagcgccgaagctgctgatttacagcgcgagcttcctgtatagcggtgtgccgagccgttttagcggcagccgtagcggcaccgacttcaccctgaccatcagcagcctgcagccggaggattttgcgacctactattgccagcaacactataccaccccgccgacctttggtcaaggcaccaaagtggaaattaagggtggcggtggcagcggtggcggtggcagcggtggcggtggcagcgaggtgcagctggttgaaagcggtggcggtctggttcaaccgggcggtagcctgcgtctgagctgcgcggcgagcggttttaacatcaaagacacctacattcactgggtgcgtcaagcgccgggcaagggtctggagtgggttgcgcgtatctatccgaccaacggctacacccgttatgcggacagcgtgaaaggtcgttttaccattagcgcggataccagcaagaacaccgcgtacctgcagatgaacagcctgcgtgcggaagataccgcggtttactattgcagccgttggggcggtgacggtttctacgcgatggattattggggccaaggcaccctggtgaccgttagcagcggcggtggcggtagcggcggtggcggtagcacccgtcaccgtcagccgcgtggttgggaacagctgggcggtagcggcggtagcggcggtagcatgaaacgtggcctgaccgtggcggttgcgggtgcggcgatcctggttgcgggcctgagcggttgcagcagcggcggtagcggcggtagcggcggcagcaatctggttccgatggtggcgaccgttcatcatcatcatcatcac

SEQ ID NO: 18 - protein, 107 aa, Artificial sequence
  Synthetic
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 19 - protein, 121 aa, Artificial sequence
  Synthetic
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 20 - protein, 243 aa, Artificial sequence
  Synthetic
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKGGGGSGGGGSGGGGSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 21 - protein, 107 aa, Artificial sequence
  Synthetic
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 22 - protein, 117 aa, Artificial sequence
  Synthetic
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 23 - protein, 239 aa, Artificial sequence
  Synthetic
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKGGGGSGGGGSGGGGSEVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 24 - protein, 107 aa, Artificial sequence [sequence omitted]

SEQ ID NO: 25 - protein, 122 aa, Artificial sequence [sequence omitted]

SEQ ID NO: 26 - protein, 244 aa, Artificial sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKGGGGSGGGGSGGGGSEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSS

SEQ ID NO: 27 - protein, 107 aa, Artificial sequence
  Synthetic
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 28 - protein, 121 aa, Artificial sequence
  Synthetic
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 29 - protein, 243 aa, Artificial sequence
  Synthetic
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 30 - protein, 105 aa, Artificial sequence
  Synthetic
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 31 - protein, 115 aa, Artificial sequence
  Synthetic
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 32 - protein, 235 aa, Artificial sequence
  Synthetic
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKGGGGSGGGGSGGGGSEVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 33 - protein, 104 aa, Artificial sequence
  Synthetic
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIK

SEQ ID NO: 34 - protein, 120 aa, Artificial sequence
  Synthetic
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSS

SEQ ID NO: 35 - protein, 239 aa, Artificial sequence
  Synthetic
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIKGGGGSGGGGSGGGGSQVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSS

SEQ ID NO: 36 - protein, 112 aa, Artificial sequence
  Synthetic
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLG

SEQ ID NO: 37 - protein, 120 aa, Artificial sequence
  Synthetic
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 38 - protein, 247 aa, Artificial sequence
  Synthetic
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGGGGGSGGGGSGGGGSQVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 39 - protein, 107 aa, Artificial sequence
  Synthetic
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIK

SEQ ID NO: 40 - protein, 189 aa, Artificial sequence
  Synthetic
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLAPSSKSTSGGTAALGCLVKDYFPEPVTVSWNSGALTSGVHTFPAVLQSSGLYSLSSVVTVPSS

SEQ ID NO: 41 - protein, 311 aa, Artificial sequence
  Synthetic
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKGGGGSGGGGSGGGGSQVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLAPSSKSTSGGTAALGCLVKDYFPEPVTVSWNSGALTSGVHTFPAVLQSSGLYSLSSVVTVPSS

SEQ ID NO: 42 - protein, 106 aa, Artificial sequence
  Synthetic
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 43 - protein, 119 aa, Artificial sequence
  Synthetic
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 44 - protein, 240 aa, Artificial sequence
  Synthetic
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGGGSGGGGSGGGGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 45 - protein, 4 aa, Artificial sequence
  Synthetic
  X (2)..(2): Any amino acid
  B (3)..(3): Selected from R and K
RXBR

SEQ ID NO: 46 - protein, 40 aa, Mycobacterium tuberculosis
MTDVSRKIRAWGRRLMIGTAAAVVLPGLVGLAGGAATAGA

SEQ ID NO: 47 - protein, 9 aa, Mycobacterium tuberculosis
TAVYAFVHA

SEQ ID NO: 48 - protein, 26 aa, Mycobacterium tuberculosis
MKIRLHTLLAVLTAAPLLLAAAGCGS

SEQ ID NO: 49 - protein, 27 aa, Mycobacterium tuberculosis
MKTGTATTRRRLLAVLIALALPGAAVA

SEQ ID NO: 50 - protein, 34 aa, Mycobacterium tuberculosis
MAAMWRRRPLSSALLSFGLLLGGLPLAAPPLAGA

SEQ ID NO: 51 - protein, 32 aa, Mycobacterium tuberculosis
MRFAQPSALSRFSALTRDWFTSTFAAPTAAQA

SEQ ID NO: 52 - protein, 25 aa, Mycobacterium tuberculosis
MLLRKGTVYVLVIRADLVNAMVAHA

SEQ ID NO: 53 - protein, 25 aa, Mycobacterium tuberculosis
MRPSRYAPLLCAMVLALAWLSAVAG

SEQ ID NO: 54 - protein, 20 aa, Mumps virus
MNAFPVICLGYAIFSSSICV

SEQ ID NO: 55 - protein, 29 aa, herpes simplex virus 7
MRPRGTPPSFLPLPVLLALAVIAAAGRAA

SEQ ID NO: 56 - protein, 24 aa, Measles virus
MGLKVNVSAIFMAVLLTLQTPTGQ

SEQ ID NO: 57 - protein, 23 aa, Measles virus
MGLRVNVSAIFMAVLLTLQTPTG

SEQ ID NO: 58 - protein, 32 aa, Corynebacterium diphtheriae
MLVRGYVVSRKLFASILIGALLGIGAPPSAHA

SEQ ID NO: 59 - protein, 22 aa, Cytomegalovirus
MESRIWCLVVCVNLCIVCLGAA

SEQ ID NO: 60 - protein, 23 aa, Cytomegalovirus
MRPGLPPYLTVFTVYLLSHLPSQ

SEQ ID NO: 61 - protein, 15 aa, Severe acute respiratory syndrome coronavirus 2
MFVFLVLLPLVSSQC

SEQ ID NO: 62 - protein, 15 aa, Severe acute respiratory syndrome coronavirus 2
MKIILFLALITLATC

SEQ ID NO: 63 - protein, 15 aa, Severe acute respiratory syndrome coronavirus 2
MKFLVFLGIITTVAA

SEQ ID NO: 64 - protein, 18 aa, Vibrio cholerae
MVKIIFVFFIFLSSFSYA

SEQ ID NO: 65 - protein, 19 aa, Vibrio cholerae
MKKIALFITASLIAGNALA

SEQ ID NO: 66 - protein, 21 aa, Vibrio cholerae
MKQTICGLAVLAALSSAPVFA

SEQ ID NO: 67 - protein, 20 aa, Vibrio cholerae
MIKLKFGVFFTVLLSSAYAH

SEQ ID NO: 68 - protein, 19 aa, Rabies virus
MVPQALLFVPLLVFPLCFG

SEQ ID NO: 69 - protein, 19 aa, Hepatitis B virus
MQLFHLCLIISCTCPTVQA

SEQ ID NO: 70 - protein, 19 aa, Hepatitis B virus
MQLFHLCLIISCSCPTVQA

SEQ ID NO: 71 - protein, 15 aa, Influenza A virus
MAIIYLILLFTAVRG

SEQ ID NO: 72 - protein, 16 aa, Influenza A virus
MEKIVLLFAIVSLVKS

SEQ ID NO: 73 - protein, 18 aa, Influenza A virus
MNTQILVFALVAIIPTNA

SEQ ID NO: 74 - protein, 42 aa, Artificial sequence
  Synthetic
MKRGLTVAVAGAAILVAGLSGCSSGGSGGSGGSNLVPMVATV

SEQ ID NO: 75 - protein, 4 aa, Artificial sequence
  Synthetic
  X (2)..(2): Any amino acid
  X (3)..(3): Any amino acid
RXXR

SEQ ID NO: 76 - protein, 4 aa, Artificial sequence
  Synthetic
RQPR

SEQ ID NO: 77 - protein, 111 aa, Artificial sequence
  Synthetic
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 78 - protein, 117 aa, Artificial sequence
  Synthetic
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 79 - protein, 243 aa, Artificial sequence
  Synthetic
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKGGGGSGGGGSGGGGSQIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 80 - protein, 111 aa, Artificial sequence [sequence omitted]

SEQ ID NO: 81 - protein, 117 aa, Artificial sequence [sequence omitted]

SEQ ID NO: 82 - protein, 243 aa, Artificial sequence
  Synthetic
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSS

SEQ ID NO: 83 - protein, 111 aa, Artificial sequence
  Synthetic
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 84 - protein, 118 aa, Artificial sequence
  Synthetic
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 85 - protein, 244 aa, Artificial sequence
  Synthetic
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKGGGGSGGGGSGGGGSQVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 86 - protein, 107 aa, Artificial sequence
  Synthetic
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 87 - protein, 121 aa, Artificial sequence
  Synthetic
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 88 - protein, 243 aa, Artificial sequence
  Synthetic
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 89 - protein, 113 aa, Artificial sequence
  Synthetic
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 90 - protein, 121 aa, Artificial sequence
  Synthetic
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 91 - protein, 249 aa, Artificial sequence
  Synthetic
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKRGGGGSGGGGSGGGGSEVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 92 - protein, 107 aa, Artificial sequence
  Synthetic
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 93 - protein, 122 aa, Artificial sequence
  Synthetic
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 94 - protein, 244 aa, Artificial sequence
  Synthetic
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKGGGGSGGGGSGGGGSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 95 - protein, 106 aa, Artificial sequence
  Synthetic
QIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELK

SEQ ID NO: 96 - protein, 122 aa, Artificial sequence
  Synthetic
EVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSS

SEQ ID NO: 97 - protein, 243 aa, Artificial sequence
  Synthetic
QIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELKGGGGSGGGGSGGGGSEVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSS

SEQ ID NO: 98 - protein, 22 aa, Homo sapiens
EPKSCDKTHTCPPCPAPELLGG

SEQ ID NO: 99 - protein, 18 aa, Homo sapiens
EPKCCVECPPCPAPPAAA

SEQ ID NO: 100 - protein, 19 aa, Homo sapiens
ESKYGPPCPPCPAPEFLGG

SEQ ID NO: 101 - protein, 70 aa, Homo sapiens
ELKTPLGDTTHTCPRCPEPKSCDTPPPCPRCPEPKSCDTPPPCPRCPEPKSCDTPPPCPRCPAPELLGGP

SEQ ID NO: 102 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 103 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 104 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 105 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 106 - protein, 102 aa, Homo sapiens
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 107 - protein, 102 aa, Homo sapiens
SVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 108 - protein, 102 aa, Homo sapiens
SVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 109 - protein, 102 aa, Homo sapiens
SVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 110 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 107 aa, Homo sapiens
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 113 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

(19) United States
(12) Patent Application Publication
(10) Pub. No.: US 2025/0101128 A1
(43) Pub. Date: Mar. 27, 2025

(54) CHIMERIC POLYPEPTIDE COMPOSITIONS AND ENCODING POLYNUCLEOTIDES THEREOF

(71) Applicant: Lior Binyamin CARMON, Ramat Hasharon (IL)

(72) Inventor: Lior Binyamin CARMON, Ramat Hasharon (IL)

(21) Appl. No.: 18/712,951

(22) PCT Filed: Nov. 25, 2021
(86) PCT No.: PCT/IL2021/051403

§ 371 Date: May 23, 2024

(51) Int. Cl. C07K 16/32; A61K 39/00; A61K 39/07; A61P 35/00; C07K 14/005

(52) U.S. Cl. CPC C07K 16/32; A61K 39/07; A61P 35/00; C07K 14/005; A61K 2039/505; C07K 2317/24; C07K 2317/622; C07K 2317/73; C07K 2317/77; C12N 2710/16122

(57) ABSTRACT

Chimeric polypeptides comprising a first subunit comprising an antigen-binding domain, a second subunit comprising at least one immunogenic peptide comprising a signal peptide and a third subunit comprising a cleavable moiety, wherein the third subunit is between the first and second subunits is provided. Nucleic acid molecules encoding the chimeric polypeptide, cells expressing the nucleic acid molecules, pharmaceutical compositions comprising the chimeric polypeptide, and methods of treating cancer by administrating the chimeric polypeptide or pharmaceutical compositions are also provided.

### CROSS REFERENCE TO RELATED APPLICATIONS

This application claims the benefit of priority of U.S. Provisional Patent Application No. 63/118,130, filed Nov. 25, 2020, the contents of which is incorporated herein by reference in its entirety.

### FIELD OF THE INVENTION

The present invention is in the field of cancer therapy.

### BACKGROUND OF THE INVENTION

Notable advances have been achieved in the treatment of cancer since the advent of immunotherapy, and immune checkpoint inhibitors (ICIs) have shown clinical benefit across a wide variety of tumor types. Nevertheless, most patients still progress on these treatments, highlighting the importance of unravelling the underlying mechanism of action (MoA) of primary resistance to immunotherapy. A well described phenomenon of non-responsiveness to ICIs, is the absence or low presence of lymphocytes in the tumor microenvironment, so-called non-inflamed or cold tumors. There are few MoAs that have the potential to turn cold tumors into so-called hot or inflamed tumors, hence increasing the tumor's responsiveness to immunotherapy. These MoAs include increasing local inflammation, neutralizing immunosuppression at the tumor site, modifying the tumor vasculature, targeting the tumor cells themselves, or increasing the frequency of anti-tumor-T cells.

Work in recent years have demonstrated that viral and bacterial induced immunity, can lead to inflamed tumors via many of the suggested MoA. Following this rationale, hosts retain memory T cells specific for previous infections throughout the entire body that can execute potent and immediate immunostimulatory functions. It has been demonstrated that virus-specific memory T cells extend their surveillance to mouse and human tumors. Reactivating these antiviral T cells can arrest growth of ICI blockade-resistant and poorly immunogenic tumors in mice after injecting adjuvant-free non-replicating viral peptides into tumors. In a different study, it was observed that FDA-approved unadjuvanted seasonal flu vaccine administered via intratumoral injection reduced tumor growth by increasing anti-tumor CD8+ T cells and decreasing regulatory B cells within the tumor. In-spite of the exciting results in the above studies, the payload being used is none immunogenic, and/or applicable only to a small population. In-addition, intratumoral injection is not a viable solution for many tumors, suggesting a novel payload delivery method is needed.

Targeted cancer immunotherapy first entered clinical practice in the late 1990s, following the approval of the B cell-depleting anti-CD20 monoclonal antibody (mAb) rituximab for the treatment of patients with B cell non-Hodgkin lymphoma. Since then, many therapeutic mAbs as well as mAb-based antibody-drug conjugates (ADCs) have been developed and are routinely used for the treatment of hematological malignancies and common solid cancers. The cytotoxic effects of classic mAbs are usually mediated by cells of the innate immune system and complement activation, both via the Fc regions of the antibodies, or by direct inhibition of growth factor receptors. ADCs are designed to deliver cytotoxic drugs to the tumor cells specifically, as an additional effector function. Strategies exploiting the potential of T cells to recognize and kill cancer cells in a targeted manner opened a novel era of cancer treatment and have led to the development of a broad immunotherapeutic armamentarium containing both drugs and more recently also molecules against specific tumor associated antigens.

Most of the epitopes from selected antigens, have a limitation in the major histocompatibility complex (MHC) coverage and consequently also T cell immune robustness. Moreover, these epitopes are not designed to deal with a tumor's tendency to reduced MHC-I via mutations in transporters associated with antigen processing (TAP), namely “TAP-deficiency”. A method of converting cold tumors to hot, and specifically methods that overcome TAP-deficiency are greatly needed.

On the contrary to the above, Carmon et al., showed that multi-epitope signal peptide domains (SPD) have a common motif but also sequence specific features. The rational of selecting entire SPD both from human and bacteria as multi-epitope long peptide (LP) vaccine candidate is based on their rich T cell epitope densities. This strategy provides for a straightforward, yet unique immunotherapeutic means of generating robust, non-toxic, diversified, combined antigen-specific CD4+/CD8+ T cell immunity, irrespective of patient MHC repertoire also in tumor TAP deficiencies to reduce membranal MHC-I expression.

There is a need in the art for specific therapies which prevent or minimize therapeutic failures due to immune resistance in cancer.

### SUMMARY OF THE INVENTION

The present invention provides chimeric polypeptides comprising a first subunit comprising an antigen-binding domain, a second subunit comprising at least one immunogenic peptide comprising a signal peptide and a third subunit comprising a cleavable moiety, wherein the third subunit is between the first and second subunits is provided. Nucleic acid molecules encoding the chimeric polypeptide are also provided. Cells expressing the nucleic acid molecules are also provided. Pharmaceutical compositions comprising the chimeric polypeptide are also provided. Methods of treating cancer by administrating the chimeric polypeptide or pharmaceutical compositions are also provided.

The present invention further provides a chimeric polypeptide comprises at least two subunits, wherein the first subunit comprises an immunoglobulin or an antigen-binding domain thereof, which has binding specificity for HER2/neu, and wherein the second subunit comprises Bacillus calmette-guérin (BCG)-derived peptide; and encoding polynucleotides thereof.

According to a first aspect, there is provided a chimeric polypeptide comprising:
a. a first subunit comprising an antigen-binding domain of a cancer surface antigen;
b. a second subunit comprising at least one immunogenic peptide comprising a signal peptide from a non-human protein or a fragment thereof capable of binding by a major histocompatibility complex (MHC) molecule; and
c. a third subunit comprising a cleavable moiety, wherein the cleavable moiety is cleaved in an endosome or lysosome of a human;
wherein the third subunit is between the first and second subunits in the polypeptide.

According to some embodiments, the first subunit comprises a single chain antibody (scFv) or single domain antibody (sdAb).

According to some embodiments, the first subunit comprises an scFv linked to an Fc region.

According to some embodiments, the cancer surface antigen is a cancer specific surface antigen which is not expressed on non-cancer cells or is significantly lower expressed on non-cancer cells than on cancer cells.

According to some embodiments, the cancer surface antigen is selected from receptor tyrosine-protein kinase ERBB2 (HER2), CD30, CD79B, Nectin 4 (NECTIN4), CD38, CD22, tumor-associated calcium signaling transducer 2 (TROP-2), epidermal growth factor receptor (EGFR), CD19, Folate Receptor alpha (FOLR1), mesothelin (MSLN), CD25, B-cell maturation antigen (BCMA), CD276, and carcinoembryonic antigen-related cell adhesion molecule 5 (CEACAM5).

According to some embodiments:
a. the cancer antigen is HER2 and the antigen binding domain comprises SEQ ID NO:18 and SEQ ID NO: 19;
b. the cancer antigen is Nectin-4 and the antigen binding domain comprises SEQ ID NO:21 and SEQ ID NO: 22;
c. the cancer antigen is CD38 and the antigen binding domain comprises SEQ ID NO:24 and SEQ ID NO: 25;
d. the cancer antigen is TROP-2 and the antigen binding domain comprises SEQ ID NO:27 and SEQ ID NO: 28;
e. the cancer antigen is EGFR and the antigen binding domain comprises SEQ ID NO:30 and SEQ ID NO: 31;
f. the cancer antigen is CD19 and the antigen binding domain comprises SEQ ID NO:33 and SEQ ID NO: 34;
g. the cancer antigen is MSLN and the antigen binding domain comprises SEQ ID NO:36 and SEQ ID NO: 37;
h. the cancer antigen is CD25 and the antigen binding domain comprises SEQ ID NO:39 and SEQ ID NO: 40;
i. the cancer antigen is CEACM5 and the antigen binding domain comprises SEQ ID NO:42 and SEQ ID NO: 43;
j. the cancer antigen is CD30 and the antigen binding domain comprises SEQ ID NO:77 and SEQ ID NO: 78;
k. the cancer antigen is CD79B and the antigen binding domain comprises SEQ ID NO:80 and SEQ ID NO: 81;
l. the cancer antigen is FLOLR1 and the antigen binding domain comprises SEQ ID NO:83 and SEQ ID NO: 84;
m. the cancer antigen is BCMA and the antigen binding domain comprises SEQ ID NO:86 and SEQ ID NO: 87;
n. the cancer antigen is CD22 and the antigen binding domain comprises SEQ ID NO:89 and SEQ ID NO: 90;
o. the cancer antigen is CD276 and the antigen binding domain comprises SEQ ID NO:92 and SEQ ID NO: 93; or
p. the cancer antigen is CD276 and the antigen binding domain comprises SEQ ID NO:95 and SEQ ID NO: 96.

According to some embodiments, the cancer antigen is HER2 and the antigen-binding domain is a HER2-specific single chain Fv (scFv).

According to some embodiments, the first subunit comprises SEQ ID NO: 1 and SEQ ID NO: 2 separated by an amino acid linker.

According to some embodiments, the first subunit consists of SEQ ID NO: 4.

According to some embodiments, the at least one immunogenic peptide comprises a sequence from a vaccine suitable for administration to humans.

According to some embodiments, the at least one immunogenic peptide comprises a sequence from a pathogen to which humans have natural immunity.

According to some embodiments, the non-human protein is selected from a viral protein, a bacterial protein and a parasite protein.

According to some embodiments, the non-human protein is selected from a tuberculosis protein, a mumps protein, a herpes simplex virus (HSV) protein, a measles protein, a diphtheria protein, a cytomegalovirus (CMV) protein, a severe acute respiratory syndrome coronavirus 2 (SARS-CoV-2) protein, a cholera protein, a rabies protein, a hepatitis B protein, and an influenza type A protein.

According to some embodiments, the non-human protein is selected from a protein provided in Table 2.

According to some embodiments, the second subunit comprises a sequence selected from SEQ ID NO: 5 and 46-73.

According to some embodiments, the second subunit comprises SEQ ID NO: 5.

According to some embodiments, the cleavable moiety is a furin-cleavable linker.

According to some embodiments, the furin-cleavable linker comprises a sequence selected from RXBR, wherein X is any amino acid and B is a positively charged amino acid selected from R and K (SEQ ID NO: 45) and SEQ ID NO: 6.

According to some embodiments, the furin-cleavable linker consists of SEQ ID NO: 6.

According to some embodiments, the second subunit further comprises a second immunogenic peptide from a non-human protein and wherein the second immunogenic peptide is C-terminal to the immunogenic peptide comprising a signal peptide or a fragment thereof.

According to some embodiments, the second immunogenic peptide comprises a sequence from a vaccine suitable for administration to humans.

According to some embodiments, the second immunogenic peptide comprises a sequence to which humans have a natural immunity.

According to some embodiments, the second immunogenic peptide consists of SEQ ID NO: 11.

According to some embodiments, the second subunit consists of the sequence MKRGLTVAVAGAAILVAGLSGCSS GGSGGSGGSNLVPMVATV (SEQ ID NO: 74).

According to some embodiments, any one of the first subunit, the second subunit and the third subunit are separated by a linker.

According to some embodiments, the linker is a flexible glycine and serine linker.

According to some embodiments, the chimeric polypeptide further comprises an N-terminal leader peptide, a C-terminal affinity tag or both.

According to some embodiments, the chimeric polypeptide comprises an amino acid sequence selected from SEQ ID NO: 12-16.

According to some embodiments, the chimeric polypeptide consists of an amino acid sequence selected from SEQ ID NO: 14 to 16 or comprising at least 90% homology thereto and being capable of binding the cancer surface antigen on a surface of a target cell and express the immunogenic peptide on a surface of the target cell.

According to another aspect, there is provided a nucleic acid molecule comprising a nucleotide sequence encoding a chimeric polypeptide of the invention.

According to some embodiments, the nucleic acid molecule further comprises at least one regulatory element operatively linked to the nucleotide sequence and capable of driving expression of the nucleotide sequence in a target cell.

According to another aspect, there is provided a host cell comprising a nucleic acid molecule of the invention.

According to another aspect, there is provided a pharmaceutical composition comprising a chimeric polypeptide of the invention and a pharmaceutically acceptable carrier, excipient or adjuvant.

According to some embodiments, the pharmaceutical is configured for systemic administration or intratumoral administration.

According to another aspect, there is provided a method of expressing an immunogenic peptide on a surface of a target cell expressing the cancer surface antigen, the method comprising contacting the target cell with a chimeric polypeptide of the invention, or a pharmaceutical composition of the invention, thereby expressing the immunogenic peptide on a surface of the target cell.

According to another aspect, there is provided a method of preventing, ameliorating, or treating a cancer expressing the cancer surface antigen in a subject in need thereof, the method comprising administrating to the subject a chimeric polypeptide of the invention, or a pharmaceutical composition of the invention, thereby preventing, ameliorating, or treating a cancer.

According to some embodiments, the method further comprises administering a vaccine to the subject before administering the chimeric polypeptide or pharmaceutical composition, wherein the vaccine comprises an amino acid sequence present in the second subunit.

According to some embodiments, the subject has already been vaccinated with a vaccine comprising an amino acid sequence present in the second subunit.

According to some embodiments, the vaccine is the Bacillus Calmette-Guerin (BCG) anti-tuberculosis vaccine and the second subunit comprises an amino acid sequence selected from SEQ ID NO: 5 and 46-53.

According to some embodiments, method further comprises treating the subject with adoptive T cell transfer or chimeric antigen receptor (CAR) therapy wherein the T cell or CAR is specific to a sequence present in the second subunit.

According to some embodiments, the cancer is a HER2-positive cancer and the first subunit is a HER2 binding domain.

According to one aspect, the present invention provides a chimeric polypeptide that is capable of binding HER2/neu, wherein the chimeric polypeptide comprises at least two subunits in any order, wherein the first subunit comprises an immunoglobulin or an antigen-binding domain thereof, which has binding specificity for HER2/neu, and wherein the second subunit comprises Bacillus calmette-guérin (BCG)-derived peptide. According to certain embodiments, the chimeric polypeptide further comprises another peptide such a CMV derived peptide. According to certain embodiments, the chimeric polypeptide further comprises at least another repeat of the BCG-derived peptide.

According to certain embodiments, the chimeric polypeptide is capable of co-stimulating T-cell responses. According to other embodiments, the immunoglobulin or an antigen-binding domain thereof is anti-HER2 mAb. According to other embodiments, the immunoglobulin or an antigen-binding domain thereof is HER2-antigen-specific single chain Fv (scFv).

According to certain embodiments, the present invention provides a method of engaging HER2/neu-positive tumor cells, comprising applying the chimeric polypeptides of the invention, or a composition comprising such chimeric polypeptide. According to other embodiments the present invention provides a method of simultaneously redirecting/co-stimulating T-cells and engaging HER2/neu-positive tumor cells, comprising applying the chimeric polypeptides of the invention, or a composition comprising such chimeric polypeptide.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1: A table of the three antigen-specific single chain Fv (scFv) constructs designed for the construction of the chimera of the present invention. Omp-C is the leader peptide used for the constructs.

FIG. 2: An SDS-PAGE gel showing the general expression levels of the three scFv variants in E. coli under different induction temperatures.

FIG. 3: An SDS-PAGE gel showing the relevant elution fractions and summarizing the results of the affinity purification process.

FIG. 4: An SDS-PAGE gel demonstrating the effect of IPTG induction on the expression of the three scFv variants.

FIG. 5: An SDS-PAGE gel showing the results of samples taken after each step of purification including the affinity purification.

FIG. 6: An SDS-PAGE gel showing the results of affinity purification on two types of nickel-chelate resin (commercial IDA-resin and DTPA-resin).

FIGS. 7A-7B: Line graphs showing the results of the ELISA assay used to determine the chimera's Her2/Neu specificity and semi-quantitative concentration with (7A) control Herceptin antibody and (7B) the scFv conjugate of the invention.

FIG. 8: A table containing the results of FACS analysis for Her2/Neu expression on the cell surface of the evaluated cell lines.

FIGS. 9A-9B: (9A) Table of membranal and intracellular levels of the control Herceptin antibody in A431 cells. (9B) Line graph of membranal and intracellular levels of the Her2ScFc-BCG-CMV construct of the invention in A431 cells.

FIGS. 10A-10B: Bar graphs demonstrates the antigen specific killing of target monocyte cells internalizing the Her2ScFc-BCG-CMV chimera and expressing the SPD 24-mer BCG peptide in (10A) a first donor and (10B) a second donor with a unique HLA repertoire.

FIG. 11: Bar chart demonstrate the antigen specific killing analysis for Her2ScFc-BCG-CMV “treated” tumor cells. *=pVal<0.002

FIG. 12: Histograms of OvCar-3 cells showing unstained cells, HLA-A2 expression and HER2 expression.

FIG. 13: Bar graph of specific and spontaneous killing in cancer cells contacted with HLA-A2.1 restricted T cells against the 9-mer CMV peptide.

FIGS. 14A-14B: Bar graph of cell killing of (14A) CT-26 mouse cancer cells expressing human HER2 in culture and (14B) CT-26 tumors grown in mice in the presence of mouse splenocytes from mice exposed to a single dose of BCG vaccine. LP=peptide only fragment. **=P-value<0.009

FIG. 15: Line graph of average tumor size over time in four groups of mice. The chimera produced a statistically significant reduction in tumor size as compared to PBS treated mice, mice that received on the vaccine and mice that received the vaccine and Her2 scFv. *=pVal<0.009

FIG. 16: Table of signal peptides, including overall MHC-I binding score, and binding affinity to specific alleles. The frequency of these alleles in the population are also shown.

### DETAILED DESCRIPTION OF THE INVENTION

The present invention, in some embodiments, provides compositions comprising a first subunit comprising an antigen-binding domain, a second subunit comprising at least one immunogenic peptide comprising a signal peptide and a third subunit comprising a cleavable moiety, wherein the third subunit is between the first and second subunits is provided. Nucleic acid molecules encoding the polypeptides of the compositions, cells expressing the nucleic acid molecules, pharmaceutical compositions comprising compositions, and methods of treating cancer by administrating the compositions or pharmaceutical compositions are also provided.

The present invention further discloses a chimeric polypeptide comprises at least two subunits, wherein the first subunit comprises an immunoglobulin or an antigen-binding domain thereof, which has binding specificity for HER2/neu, and wherein the second subunit comprises Bacillus calmette-guérin (BCG)-derived peptide.

The invention is based on the surprising finding that immunogenic signal peptides, preferably signal peptides with broad HLA repertoires, once delivered to the inside of cells are efficiently brought to the cell surface. Using other short peptides does not induce a robust response in all patients, while using very long sequences is less specific and can lead to antibody production that many limit the overall use of the product. Further, most sequences are not helpful in dealing with TAP-deficiency associated with many solid tumors. In contrast, immunogenic signal peptides can be efficiently targeted to cancers, internalized and released by the use of a single polypeptide chain binding domain that targets cancer surface antigens and which contains a C-terminal cleavage site that allows for release of the more C-terminal signal peptide. Thus, the binding domain produces cancer cell targeting and internalization upon antigen binding. Once, in the endosomal/lysosomal compartments the cleavage site releases the signal peptide and any C-terminal sequence. Finally, the signal peptide mediates HLA binding, ER and Golgi entrance and shuttling to the cell surface. Once the immunogenic peptide is displayed on the cell surface (alone with any other C-terminal sequences) this surface display renders the cancer cell detectable by immune cells and targets it for immune cell killing. In this way, the molecules of the invention can convert a cold tumor, one that avoids immune surveillance, into a hot one.

### Chimeric Polypeptides

According to a first aspect, there is provided a composition comprising a first subunit and a second subunit.

According to another aspect, there is provided a chimeric polypeptide comprising a first subunit and a second subunit.

In some embodiments, the composition comprises the chimeric polypeptide of the invention. In some embodiments, the first subunit and the second subunit are on different polypeptides. In some embodiments, the first subunit is a protein complex. In some embodiments, the first subunit is comprised on a plurality of polypeptides. In some embodiments, one polypeptide of the plurality comprises the second subunit. In some embodiments, a polypeptide of the protein complex comprises the second subunit. In some embodiments, a polypeptide comprises a third subunit. In some embodiments, a polypeptide comprising the second subunit further comprises a third subunit.

In some embodiments, the first subunit comprises an antigen-binding region. In some embodiments, the first subunit comprises an antigen-binding motif. In some embodiments, the first subunit comprises an antigen-binding structure. In some embodiments, the first subunit comprises an antigen-binding domain. In some embodiments, the antigen-binding domain comprises an aptamer. In some embodiments, the composition comprises an aptamer. In some embodiments, the aptamer is a peptide aptamer. In some embodiments, the antigen binding domain is a binding domain of an antibody. In some embodiments, the antigen binding domain is an antibody. In some embodiments, a chain of the antibody comprises the second subunit. In some embodiments, the second subunit is at the C-terminal end of a chain of the antibody. In some embodiments, the chain is the heavy chain. In some embodiments, the chain is the light chain. In some embodiments, the antigen binding domain is a binding domain of an immunoglobulin. In some embodiments, the antigen binding domain is a single chain antibody (scFv). In some embodiments, the scFv comprises an Fc domain. In some embodiments, the scFv is devoid of an Fc domain. In some embodiments, the second subunit is linked to the scFv. In some embodiments, the second subunit is linked to the Fc domain. In some embodiments, the antigen binding domain is a single domain antibody sdAb. In some embodiments, the antigen binding domain is a single polypeptide chain. Methods of converting antibody heavy and light chains into scFvs are well known in the art and may be used in order to convert any antibody known in the art into a single chain to be used in a polypeptide of the invention. In some embodiments, an scFv is generated by linking the variable domain of a heavy chain of an antibody to the variable domain of the light chain of the antibody. In some embodiments, an scFv is generated by the linking the light chain variable domain to the N-terminus of the heavy chain. In some embodiments, the linking is via a linker. In some embodiments, the linker is a peptide linker. In some embodiments, the linker is an amino acid linker. In some embodiments, the linker is a flexible linker. In some embodiments, the linker is a rigid linker. In some embodiments, the linker is a glycine-serine (GS) linker. In some embodiments, the linker comprises or consists of (GGGGS)x, wherein x is a integer. In some embodiments, x is selected from 1 to 5. In some embodiments, x is 1. In some embodiments, x is 2. In some embodiments, x is 3. In some embodiments, x is 4. In some embodiments, x is 5. In some embodiments, the linker comprises GGGGSGGGGSGGGGS (SEQ ID NO: 3). In some embodiments, the linker consists of SEQ ID NO: 3. In some embodiments, the linker comprises GGGGSGGGGS (SEQ ID NO: 7). In some embodiments, the linker consists of SEQ ID NO: 7. In some embodiments, the linker comprises (GGS)x, wherein x is an integer. In some embodiments, the linker comprises GGSGGSGGS (SEQ ID NO: 8). In some embodiments, the linker consists of SEQ ID NO: 8.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (scFv), dimeric variable region (Diabody), single domain antibodies (sdAbs, e.g., VHHs and camelid or shark antibodies) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜ Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the Fc domain is the constant region of the heavy chain. In some embodiments, the Fc domain is an Ig heavy chain. In some embodiments, the Fc domain is an IgG heavy chain. In some embodiments, the Fc domain is an IgM heavy chain. In some embodiments, IgG is IgG1. In some embodiments, IgG is IgG2. In some embodiments, IgG is IgG3. In some embodiments, IgG is IgG4. In some embodiments, IgG is selected from IgG1, IgG2, IgG3 and IgG4. In some embodiments, the Fc is devoid of the hinge region. In some embodiments, the Fc does not comprise the hinge region. In some embodiments, the Fc comprises the hinge region. In some embodiments, the Fc is devoid of the CH1 domain. In some embodiments, the Fc does not comprise the CH1 domain. In some embodiments, the Fc comprises the CH1 domain. In some embodiments, the Fc comprises a CH2 domain. In some embodiments, the Fc comprises a CH3 domain. In some embodiments, the second subunit is linked to the C-terminus of the Fc. In some embodiments, the second subunit is linked to the C-terminus of the CH3 domain.

In some embodiments, the hinge domain comprises the amino acid sequence EPKSCDKTHTCPPCPAPELLGG (SEQ ID NO: 98). In some embodiments, the hinge domain consists of the amino acid sequence of SEQ ID NO: 98. In some embodiments, the IgG1 hinge comprises or consists of SEQ ID NO: 98. In some embodiments, the hinge domain comprises the amino acid sequence EPKCCVECPPCPAPPAAA (SEQ ID NO: 99). In some embodiments, the hinge domain consists of the amino acid sequence of SEQ ID NO: 99. In some embodiments, the IgG2 hinge comprises or consists of SEQ ID NO: 99. In some embodiments, the hinge domain comprises the amino acid sequence ESKYGPPCPPCPAPEFLGG (SEQ ID NO: 100). In some embodiments, the hinge domain consists of the amino acid sequence of SEQ ID NO: 100. In some embodiments, the IgG4 hinge comprises or consists of SEQ ID NO: 100. In some embodiments, the hinge domain comprises the amino acid sequence ELKTPLGDTTHTCPRCPEPKSCDTPPPCPRCPEPKSCDTPPPCPRCPEPKSCDTPP PCPRCPAPELLGGP (SEQ ID NO: 101). In some embodiments, the hinge domain consists of the amino acid sequence of SEQ ID NO: 101. In some embodiments, the IgG3 hinge comprises or consists of SEQ ID NO: 101.

In some embodiments, a CH1 domain comprises of the amino acid sequence ASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKV (SEQ ID NO: 102). In some embodiments, an CH1 domain consists of SEQ ID NO: 102. In some embodiments, SEQ ID NO: 102 is the IgG1 CH1 domain. In some embodiments, a CH1 domain comprises of the amino acid sequence ASTKGPSVFPLAPCSRSTSESTAALGCLVKDYFPEPVTVSWNSGALTSGVHTFP AVLQSSGLYSLSSVVTVPSSNFGTQTYTCNVDHKPSNTKVDKTV (SEQ ID NO: 103). In some embodiments, an Ig CH1 domain consists of SEQ ID NO: 103. In some embodiments, SEQ ID NO: 103 is the IgG2 CH1 domain. In some embodiments, a CH1 domain comprises of the amino acid sequence ASTKGPSVFPLAPCSRSTSGGTAALGCLVKDYFPEPVTVSWNSGALTSGVHTF PAVLQSSGLYSLSSVVTVPSSSLGTQTYTCNVNHKPSNTKVDKRV (SEQ ID NO: 104). In some embodiments, a CH1 domain consists of SEQ ID NO: 104. In some embodiments, SEQ ID NO: 104 is the IgG3 CH1 domain. In some embodiments, a CH1 domain comprises the amino acid sequence ASTKGPSVFPLAPCSRSTSESTAALGCLVKDYFPEPVTVSWNSGALTSGVHTFP AVLQSSGLYSLSSVVTVPSSSLGTKTYTCNVDHKPSNTKVDKRV (SEQ ID NO: 105). In some embodiments, a CH1 domain consists of SEQ ID NO: 105. In some embodiments, SEQ ID NO: 105 is the IgG4 CH1 domain.

In some embodiments, a CH2 domain comprises the amino acid sequence SVFLFPPKPKDTLMISRTPEVTCVVVDVSHEDPEVKFNWYVDGVEVHNAKTK PREEQYNSTYRVVSVLTVLHQDWLNGKEYKCKVSNKALPAPIEKTISKAK (SEQ ID NO: 106). In some embodiments, the CH2 domain consists of SEQ ID NO: 106. In some embodiments, SEQ ID NO: 106 is the IgG1 CH2 domain. In some embodiments, a CH2 domain comprises the amino acid sequence SVFLFPPKPKDTLMISRTPEVTCVVVDVSHEDPEVQFNWYVDGVEVHNAKTK PREEQFNSTFRVVSVLTVVHQDWLNGKEYKCKVSNKGLPAPIEKTISKTK (SEQ ID NO: 107). In some embodiments, the CH2 domain consists of SEQ ID NO: 107. In some embodiments, SEQ ID NO: 107 is the IgG2 CH2 domain. In some embodiments, a CH2 domain comprises the amino acid sequence SVFLFPPKPKDTLMISRTPEVTCVVVDVSQEDPEVQFNWYVDGVEVHNAKTK PREEQFNSTYRVVSVLTVLHQDWLNGKEYKCKVSNKGLPSSIEKTISKAK (SEQ ID NO: 108). In some embodiments, the CH2 domain consists of SEQ ID NO: 108. In some embodiments, SEQ ID NO: 108 is the IgG4 CH2 domain. In some embodiments, a CH2 domain comprises the amino acid sequence SVFLFPPKPKDTLMISRTPEVTCVVVDVSHEDPEVQFKWYVDGVEVHNAKTK PREEQYNSTFRVVSVLTVLHQDWLNGKEYKCKVSNKALPAPIEKTISKTK (SEQ ID NO: 109). In some embodiments, the CH2 domain consists of SEQ ID NO: 109. In some embodiments, SEQ ID NO: 109 is the IgG3 CH2 domain.

In some embodiments, a CH3 domain comprises the amino acid sequence GQPREPQVYTLPPSREEMTKNQVSLTCLVKGFYPSDIAVEWESNGQPENNYKT TPPVLDSDGSFFLYSKLTVDKSRWQQGNVFSCSVMHEALHNHYTQKSLSLSP GK (SEQ ID NO: 110). In some embodiments, a CH3 domain comprises the amino acid sequence GQPREPQVYTLPPSRDELTKNQVSLTCLVKGFYPSDIAVEWESNGQPENNYKT TPPVLDSDGSFFLYSKLTVDKSRWQQGNVFSCSVMHEALHNHYTQKSLSLSP GK (SEQ ID NO: 111). In some embodiments, the CH3 domain consists of SEQ ID NO: 110. In some embodiments, the CH3 domain consists of SEQ ID NO: 111. In some embodiments, SEQ ID NO: 110 is the IgG1 CH3 domain. In some embodiments, SEQ ID NO: 111 is the IgG1 CH3 domain. In some embodiments, the SEQ ID NO: 110 sequence is the sequence found predominantly is humans of European and American descent. In some embodiments, SEQ ID NO: 111 is the sequence found predominantly in humans of Asian descent. In some embodiments, a CH3 domain comprises the amino acid sequence GQPREPQVYTLPPSREEMTKNQVSLTCLVKGFYPSDISVEWESNGQPENNYKT TPPMLDSDGSFFLYSKLTVDKSRWQQGNVFSCSVMHEALHNHYTQKSLSLSP GK (SEQ ID NO: 112). In some embodiments, the CH3 domain consists of SEQ ID NO: 112. In some embodiments, SEQ ID NO: 112 is the IgG2 CH3 domain. In some embodiments, a CH3 domain comprises the amino acid sequence GQPREPQVYTLPPSQEEMTKNQVSLTCLVKGFYPSDIAVEWESNGQPENNYK TTPPVLDSDGSFFLYSRLTVDKSRWQEGNVFSCSVMHEALHNHYTQKSLSLSL GK (SEQ ID NO: 113). In some embodiments, the CH3 domain consists of SEQ ID NO: 113. In some embodiments, SEQ ID NO: 113 is the IgG4 CH3 domain. In some embodiments, a CH3 domain comprises the amino acid sequence GQPREPQVYTLPPSREEMTKNQVSLTCLVKGFYPSDIAVEWESSGQPENNYNT TPPMLDSDGSFFLYSKLTVDKSRWQQGNIFSCSVMHEALHNRFTQKSLSLSPG K (SEQ ID NO: 114). In some embodiments, the CH3 domain consists of SEQ ID NO: 114. In some embodiments, SEQ ID NO: 114 is the IgG3 CH3 domain.

In some embodiments, the antigen binding domain binds to a cancer antigen. In some embodiments, a cancer antigen is a cancer associated antigen. In some embodiments, a cancer antigen is a cancer specific antigen. In some embodiments, the antigen binding domain is specific to the cancer antigen. As used herein, the term “specific” refers to binding to a target to the exclusion of significant binding to other targets. An antibody that significantly binds more than one target is not considered specific. In some embodiments, the antigen binding domain is specific. In some embodiments, the antigen binding domain is specific to the cancer antigen. In some embodiments, the antigen binding domain is specific to the cancer antigen when it is on a cell surface. In some embodiments, on a cell surface is in a plasma membrane. In some embodiments, on a cell surface is on a plasma membrane. In some embodiments, the cancer antigen is a cancer surface antigen. In some embodiments, the antigen binding domain binds to an extracellular fragment of the cancer antigen. In some embodiments, the extracellular fragment is an extracellular peptide. In some embodiments, the extracellular fragment is the extracellular domain. It will be understood by a skilled artisan that as the antigen binding domain binds to the antigen while on the surface of a cancer cells it will need to bind to a region of the target protein that is extracellular.

As used herein, the term “cancer antigen” refers to a peptide that is uniquely present on cancer cells to the exclusion of non-cancer cells (i.e., is not expressed on non-cancer cells and is therefore cancer specific) or a peptide that is higher expressed on cancer cells than on non-cancer cells (i.e., lower expressed on non-cancer cells than cancer cells and is therefore cancer associated). In some embodiments, higher or lower is significantly higher or lower. In some embodiments, higher is at least 5, 10, 15, 20, 25, 30, 35, 40, 45, 50, 55, 60, 65, 70, 75, 80, 85, 90, 95, 100, 110, 125, 150, 175, 200, 250, 300, 350, 400, 450, or 500% higher. Each possibility represents a separate embodiment of the invention. In some embodiments, higher is at least 100% higher. In some embodiments, lower is at least 5, 10, 15, 20, 25, 30, 35, 40, 45, 50, 55, 60, 65, 70, 75, 80, 85, 90, 95, 97, 99 or 100% lower. Each possibility represents a separate embodiment of the invention. In some embodiments, lower is at least 50% lower.

In some embodiments, the cancer antigen is a mammalian protein. In some embodiments, the mammal is a mouse. In some embodiments, the mammal is a human. In some embodiments, the cancer antigen is selected from a protein provided in Table 1. In some embodiments, the cancer antigen is selected from receptor tyrosine-protein kinase ERBB2 (HER2), CD30, CD79B, Nectin 4 (NECTIN4), CD38, CD22, tumor-associated calcium signaling transducer 2 (TROP-2), epidermal growth factor receptor (EGFR), CD19, Folate Receptor alpha (FOLR1), mesothelin (MSLN), CD25, B-cell maturation antigen (BCMA), CD276, and carcinoembryonic antigen-related cell adhesion molecule 5 (CEACAM5). In some embodiments, the cancer antigen is HER2.

As used herein, the term “HER2” or “HER2/neu” refers to receptor tyrosine-protein kinase ERBB2 which is also known asHER2. Her-2 or HER2/neu is also known as “erbB-2”, “c-neu”, or “p185”. In some embodiments, HER2 is human HER2. The amino acid sequence of human HER2 can be found in UniProt P04626. In some embodiments, the antigen binding domain binds to an extracellular region of HER2. In some embodiments, the antigen binding domain is specific to HER2.

The binding affinity, or binding specificity of n antigen binding domain of the disclosure or a fusion polypeptide thereof to a selected target (e.g., HER2/neu), can be measured by any method known to those skilled in the art. Such methods include, but are not limited to, fluorescence titration, competition ELISA, calorimetric methods, such as isothermal titration calorimetry (ITC), and surface plasmon resonance (BIAcore). Such methods are well established in the art and examples thereof are also detailed below.

The term “fragment” as used herein in connection with the disclosure relates to proteins or peptides derived from full-length mature human protein that are N-terminally and/or C-terminally shortened, i.e., lacking at least one of the N-terminal and/or C-terminal amino acids. Such fragments may include at least 10, at least 20, at least 25, at least 30, at least 40, at least 50, at least 60, at least 70, at least 75, at least 80, at least 90, at least 100 or more consecutive amino acids of the primary sequence of the mature protein and are usually detectable in an immunoassay of the mature protein. In general, the term “fragment”, as used herein with respect to the corresponding protein of the disclosure or of the combination according to the disclosure or of a fusion protein described herein, relates to N-terminally and/or C-terminally shortened protein or peptide ligands, which retain the capability of the full length ligand to be recognized and/or bound by a protein according to the disclosure.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, then first subunit comprises the antigen binding domain. In some embodiments, the first subunit consists of the antigen binding domain. In some embodiments, the first subunit comprises a sequence identical to that of the antigen binding domain. In some embodiments, the first subunit comprises a sequence with at least 70, 75, 80, 85, 90, 92, 95, 97, 99 or 100% identity to the antigen binding domain. Each possibility represents a separate embodiment of the invention. In some embodiments, the first subunit comprises a sequence with at least 70, 75, 80, 85, 90, 92, 95, 97, 99 or 100% homology to the antigen binding domain. Each possibility represents a separate embodiment of the invention. In some embodiments, the first subunit comprises a variant of the antigen binding domain. In some embodiments, a sequence that is not identical to the antigen binding domain retains antigen binding. In some embodiments, retaining comprises retaining a not reduced antigen binding. In some embodiments, retaining comprises retaining specificity.

“Identity” is a property of sequences that measures their similarity or relationship. The term “sequence identity” or “identity” as used in the present disclosure means the percentage of pair-wise identical residues-following (homologous) alignment of a sequence of a polypeptide of the disclosure with a sequence in question—with respect to the number of residues in the longer of these two sequences. Sequence identity is measured by dividing the number of identical amino acid residues by the total number of residues and multiplying the product by 100.

The term “homology” is used herein in its usual meaning and includes identical amino acids as well as amino acids which are regarded to be conservative substitutions (for example, exchange of a glutamate residue by an aspartate residue) at equivalent positions in the linear amino acid sequence of a polypeptide of the disclosure.

The term “variant” as used in the present disclosure relates to derivatives of a protein or peptide that include modifications of the amino acid sequence, for example by substitution, deletion, insertion or chemical modification. In some embodiments, the variant or homolog does not comprise reduced functionality of the protein or peptide. In some embodiments, functionality is binding. Such variants include proteins, wherein one or more amino acids have been replaced by their respective D-stereoisomers or by amino acids other than the naturally occurring 20 amino acids, such as, for example, ornithine, hydroxyproline, citrulline, homoserine, hydroxylysine, norvaline. However, such substitutions may also be conservative, i.e., an amino acid residue is replaced with a chemically similar amino acid residue. Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan.

The term “position” when used in accordance with the disclosure means the position of either an amino acid within an amino acid sequence depicted herein or the position of a nucleotide within a nucleic acid sequence depicted herein.

In some embodiments, the antigen binding domain comprises or consists of an amino acid sequence provided in Table 1. In some embodiments, the antigen is HER2 and the antigen binding domain comprises or consists of SEQ ID NO: 1 and SEQ ID NO: 2, or homologs or variants thereof that retain binding to HER2, separated by a linker. In some embodiments, the antigen is HER2 and the antigen binding domain comprises or consists of SEQ ID NO: 1 and SEQ ID NO: 2, separated by a linker. In some embodiments, the antigen is HER2 and the antigen binding domain comprises or consists of SEQ ID NO: 4, or a homology or variant thereof that retains binding to HER2. In some embodiments, the antigen is HER2 and the antigen binding domain comprises SEQ ID NO: 4. In some embodiments, the antigen is HER2 and the antigen binding domain consists of SEQ ID NO: 4. In some embodiments, binding is specific binding. In some embodiments, the antigen is HER2 and the antigen binding domain comprises or consists of SEQ ID NO: 18 and SEQ ID NO: 19, or homologs or variants thereof that retain binding to HER2, separated by a linker. In some embodiments, the antigen is HER2 and the antigen binding domain comprises or consists of SEQ ID NO: 18 and SEQ ID NO: 19, separated by a linker. In some embodiments, the antigen is HER2 and the antigen binding domain comprises or consists of SEQ ID NO: 20, or a homology or variant thereof that retains binding to HER2. In some embodiments, the antigen is HER2 and the antigen binding domain comprises SEQ ID NO: 20. In some embodiments, the antigen is HER2 and the antigen binding domain consists of SEQ ID NO: 20.

In some embodiments, the antigen is Nectin-4. In some embodiments, the antigen is Nectin-4 and the antigen binding domain comprises or consists of SEQ ID NO: 21 and SEQ ID NO: 22, or homologs or variants thereof that retain binding to NECTIN-4, separated by a linker. In some embodiments, the antigen is NECTIN-4 and the antigen binding domain comprises or consists of SEQ ID NO: 21 and SEQ ID NO: 22, separated by a linker. In some embodiments, the antigen is NECTIN-4 and the antigen binding domain comprises or consists of SEQ ID NO: 23, or a homology or variant thereof that retains binding to NECTIN-4. In some embodiments, the antigen is NECTIN-4 and the antigen binding domain comprises SEQ ID NO: 23. In some embodiments, the antigen is NECTIN-4 and the antigen binding domain consists of SEQ ID NO: 23.

In some embodiments, the antigen is CD38. In some embodiments, the antigen is CD38 and the antigen binding domain comprises or consists of SEQ ID NO: 24 and SEQ ID NO: 25, or homologs or variants thereof that retain binding to CD38, separated by a linker. In some embodiments, the antigen is CD38 and the antigen binding domain comprises or consists of SEQ ID NO: 24 and SEQ ID NO: 25, separated by a linker. In some embodiments, the antigen is CD38 and the antigen binding domain comprises or consists of SEQ ID NO: 26, or a homology or variant thereof that retains binding to CD38. In some embodiments, the antigen is CD38 and the antigen binding domain comprises SEQ ID NO: 26. In some embodiments, the antigen is CD38 and the antigen binding domain consists of SEQ ID NO: 26.

In some embodiments, the antigen is tumor-associated calcium signaling transducer 2 (TROP-2). In some embodiments, the antigen is TROP-2 and the antigen binding domain comprises or consists of SEQ ID NO: 27 and SEQ ID NO: 28, or homologs or variants thereof that retain binding to TROP-2, separated by a linker. In some embodiments, the antigen is TROP-2 and the antigen binding domain comprises or consists of SEQ ID NO: 27 and SEQ ID NO: 28, separated by a linker. In some embodiments, the antigen is TROP-2 and the antigen binding domain comprises or consists of SEQ ID NO: 29, or a homology or variant thereof that retains binding to TROP-2. In some embodiments, the antigen is TROP-2 and the antigen binding domain comprises SEQ ID NO: 29. In some embodiments, the antigen is TROP-2 and the antigen binding domain consists of SEQ ID NO: 29.

In some embodiments, the antigen is epidermal growth factor receptor (EGFR). In some embodiments, the antigen is EGFR and the antigen binding domain comprises or consists of SEQ ID NO: 30 and SEQ ID NO: 31, or homologs or variants thereof that retain binding to EGFR, separated by a linker. In some embodiments, the antigen is EGFR and the antigen binding domain comprises or consists of SEQ ID NO: 30 and SEQ ID NO: 31, separated by a linker. In some embodiments, the antigen is EGFR and the antigen binding domain comprises or consists of SEQ ID NO: 32, or a homology or variant thereof that retains binding to EGFR. In some embodiments, the antigen is EGFR and the antigen binding domain comprises SEQ ID NO: 32. In some embodiments, the antigen is EGFR and the antigen binding domain consists of SEQ ID NO: 32.

In some embodiments, the antigen is CD19. In some embodiments, the antigen is CD19 and the antigen binding domain comprises or consists of SEQ ID NO: 33 and SEQ ID NO: 34, or homologs or variants thereof that retain binding to CD19, separated by a linker. In some embodiments, the antigen is CD19 and the antigen binding domain comprises or consists of SEQ ID NO: 33 and SEQ ID NO: 34, separated by a linker. In some embodiments, the antigen is CD19 and the antigen binding domain comprises or consists of SEQ ID NO: 35, or a homology or variant thereof that retains binding to CD19. In some embodiments, the antigen is CD19 and the antigen binding domain comprises SEQ ID NO: 35. In some embodiments, the antigen is CD19 and the antigen binding domain consists of SEQ ID NO: 35.

In some embodiments, the antigen is mesothelin (MSLN). In some embodiments, the antigen is MSLN and the antigen binding domain comprises or consists of SEQ ID NO: 36 and SEQ ID NO: 37, or homologs or variants thereof that retain binding to MSLN, separated by a linker. In some embodiments, the antigen is MSLN and the antigen binding domain comprises or consists of SEQ ID NO: 36 and SEQ ID NO: 37, separated by a linker. In some embodiments, the antigen is MSLN and the antigen binding domain comprises or consists of SEQ ID NO: 38, or a homology or variant thereof that retains binding to MSLN. In some embodiments, the antigen is MSLN and the antigen binding domain comprises SEQ ID NO: 38. In some embodiments, the antigen is MSLN and the antigen binding domain consists of SEQ ID NO: 38.

In some embodiments, the antigen is CD25. In some embodiments, the antigen is CD25 and the antigen binding domain comprises or consists of SEQ ID NO: 39 and SEQ ID NO: 40, or homologs or variants thereof that retain binding to CD25, separated by a linker. In some embodiments, the antigen is CD25 and the antigen binding domain comprises or consists of SEQ ID NO: 39 and SEQ ID NO: 40, separated by a linker. In some embodiments, the antigen is CD25 and the antigen binding domain comprises or consists of SEQ ID NO: 41, or a homology or variant thereof that retains binding to CD25. In some embodiments, the antigen is CD25 and the antigen binding domain comprises SEQ ID NO: 41. In some embodiments, the antigen is CD25 and the antigen binding domain consists of SEQ ID NO: 41.

In some embodiments, the antigen is carcinoembryonic antigen-related cell adhesion molecule 5 (CEACAM5). In some embodiments, the antigen is CEACAM5 and the antigen binding domain comprises or consists of SEQ ID NO: 42 and SEQ ID NO: 43, or homologs or variants thereof that retain binding to CEACAM5, separated by a linker. In some embodiments, the antigen is CEACAM5 and the antigen binding domain comprises or consists of SEQ ID NO: 42 and SEQ ID NO: 43, separated by a linker. In some embodiments, the antigen is CEACAM5 and the antigen binding domain comprises or consists of SEQ ID NO: 44, or a homology or variant thereof that retains binding to CEACAM5. In some embodiments, the antigen is CEACAM5 and the antigen binding domain comprises SEQ ID NO: 44. In some embodiments, the antigen is CEACAM5 and the antigen binding domain consists of SEQ ID NO: 44.

In some embodiments, the antigen is CD30. In some embodiments, the antigen is CD30 and the antigen binding domain comprises or consists of SEQ ID NO: 77 and SEQ ID NO: 78, or homologs or variants thereof that retain binding to CD30, separated by a linker. In some embodiments, the antigen is CD30 and the antigen binding domain comprises or consists of SEQ ID NO: 77 and SEQ ID NO: 78, separated by a linker. In some embodiments, the antigen is CD30 and the antigen binding domain comprises or consists of SEQ ID NO: 79, or a homology or variant thereof that retains binding to CD30. In some embodiments, the antigen is CD30 and the antigen binding domain comprises SEQ ID NO: 79. In some embodiments, the antigen is CD30 and the antigen binding domain consists of SEQ ID NO: 79.

In some embodiments, the antigen is CD79B. In some embodiments, the antigen is CD79B and the antigen binding domain comprises or consists of SEQ ID NO: 80 and SEQ ID NO: 81, or homologs or variants thereof that retain binding to CD79B, separated by a linker. In some embodiments, the antigen is CD79B and the antigen binding domain comprises or consists of SEQ ID NO: 80 and SEQ ID NO: 81, separated by a linker. In some embodiments, the antigen is CD79B and the antigen binding domain comprises or consists of SEQ ID NO: 82, or a homology or variant thereof that retains binding to CD79B. In some embodiments, the antigen is CD79B and the antigen binding domain comprises SEQ ID NO: 82. In some embodiments, the antigen is CD79B and the antigen binding domain consists of SEQ ID NO: 82.

In some embodiments, the antigen is CD22. In some embodiments, the antigen is CD22 and the antigen binding domain comprises or consists of SEQ ID NO: 89 and SEQ ID NO: 90, or homologs or variants thereof that retain binding to CD22, separated by a linker. In some embodiments, the antigen is CD22 and the antigen binding domain comprises or consists of SEQ ID NO: 89 and SEQ ID NO: 90, separated by a linker. In some embodiments, the antigen is CD22 and the antigen binding domain comprises or consists of SEQ ID NO: 91, or a homology or variant thereof that retains binding to CD22. In some embodiments, the antigen is CD22 and the antigen binding domain comprises SEQ ID NO: 91. In some embodiments, the antigen is CD22 and the antigen binding domain consists of SEQ ID NO: 91.

In some embodiments, the antigen is folate receptor alpha (FOLR1). In some embodiments, the antigen is FOLR1 and the antigen binding domain comprises or consists of SEQ ID NO: 83 and SEQ ID NO: 84, or homologs or variants thereof that retain binding to FOLR1, separated by a linker. In some embodiments, the antigen is FOLR1 and the antigen binding domain comprises or consists of SEQ ID NO: 83 and SEQ ID NO: 84, separated by a linker. In some embodiments, the antigen is FOLR1 and the antigen binding domain comprises or consists of SEQ ID NO: 85, or a homology or variant thereof that retains binding to FOLR1. In some embodiments, the antigen is FOLR1 and the antigen binding domain comprises SEQ ID NO: 85. In some embodiments, the antigen is FOLR1 and the antigen binding domain consists of SEQ ID NO: 85.

In some embodiments, the antigen is B-cell maturation antigen (BCMA). In some embodiments, the antigen is BCMA and the antigen binding domain comprises or consists of SEQ ID NO: 86 and SEQ ID NO: 87, or homologs or variants thereof that retain binding to BCMA, separated by a linker. In some embodiments, the antigen is BCMA and the antigen binding domain comprises or consists of SEQ ID NO: 86 and SEQ ID NO: 87, separated by a linker. In some embodiments, the antigen is BCMA and the antigen binding domain comprises or consists of SEQ ID NO: 88, or a homology or variant thereof that retains binding to BCMA. In some embodiments, the antigen is BCMA and the antigen binding domain comprises SEQ ID NO: 88. In some embodiments, the antigen is BCMA and the antigen binding domain consists of SEQ ID NO: 88.

In some embodiments, the antigen is CD276. CD276 is also known as B7-H3. In some embodiments, the antigen is CD276 and the antigen binding domain comprises or consists of SEQ ID NO: 92 and SEQ ID NO: 93, or homologs or variants thereof that retain binding to CD276, separated by a linker. In some embodiments, the antigen is CD276 and the antigen binding domain comprises or consists of SEQ ID NO: 92 and SEQ ID NO: 93, separated by a linker. In some embodiments, the antigen is CD276 and the antigen binding domain comprises or consists of SEQ ID NO: 94, or a homology or variant thereof that retains binding to CD276. In some embodiments, the antigen is CD276 and the antigen binding domain comprises SEQ ID NO: 94. In some embodiments, the antigen is CD276 and the antigen binding domain consists of SEQ ID NO: 94. In some embodiments, the antigen is CD276 and the antigen binding domain comprises or consists of SEQ ID NO: 95 and SEQ ID NO: 96, or homologs or variants thereof that retain binding to CD276, separated by a linker. In some embodiments, the antigen is CD276 and the antigen binding domain comprises or consists of SEQ ID NO: 95 and SEQ ID NO: 96, separated by a linker. In some embodiments, the antigen is CD276 and the antigen binding domain comprises or consists of SEQ ID NO: 97, or a homology or variant thereof that retains binding to CD276. In some embodiments, the antigen is CD276 and the antigen binding domain comprises SEQ ID NO: 97. In some embodiments, the antigen is CD276 and the antigen binding domain consists of SEQ ID NO: 97.

In some embodiments, the second subunit comprises at least one immunogenic peptide. As used herein, the term “immunogenic” refers to an amino acid sequence that induces an immune response. In some embodiments, the immune response is a T cell response. In some embodiments, the immune response is a lymphocyte response. In some embodiments, the immune response is a cytotoxic response. In some embodiments, the immunogenic peptide is a peptide from a non-human source. In some embodiments, the immunogenic peptide is a peptide from a non-human protein. In some embodiments, the non-human is a bacterium. In some embodiments, the non-human is a virus. In some embodiments, the non-human protein is a bacterial or viral protein. In some embodiments, the non-human is a parasite. In some embodiments, the non-human protein is a bacterial or parasite protein. In some embodiments, the non-human protein is a viral or parasite protein. In some embodiments, the non-human protein is a bacterial, viral or parasite protein. In some embodiments, the non-human is a non-human organism for which there is a vaccine. In some embodiments, the non-human is a non-human organism for which the subject has a natural immunity. In some embodiments, the non-human is a non-human organism for which a percentage of the population has a natural immunity. In some embodiments, the percentage is at least 5, 10, 15, 20, 25, 30, 35, 40, 45, 50, 55, 60, 65, 70, 75, 80, 85, 90, 95, 97, 99 or 100%. Each possibility represents a separate embodiment of the invention.

In some embodiments, the non-human protein is a surface protein. In some embodiments, the non-human protein is a receptor. In some embodiments, the non-human protein is a secreted protein. In some embodiments, the non-human protein comprises a signal peptide. In some embodiments, the immunogenic peptide comprises a signal peptide or a fragment thereof. In some embodiments, the immunogenic peptide consists of a signal peptide or a fragment thereof. In some embodiments, the immunogenic peptide comprises or consists of a signal peptide. In some embodiments, a signal peptide is a leader peptide.

Proteins directed into the secretory pathway use amino-terminal signal peptides to interact with the translation machinery. The translocation of secretory proteins across intracellular membranes and final localization are mediated by signal peptides (SP) which are ‘address tags’ contained within their amino acid sequences. Signal peptides, comprising the N-terminal 15-60 amino acids of proteins, are necessary for the translocation across the membrane on the secretory pathway and thus universally control the entry of all proteins both in eukaryotes and prokaryotes to the secretory pathway. Signal peptides generally consist of three parts: an N-terminal region of differing length, which usually comprises positively charged amino acids; a hydrophobic domain; and a short carboxy-terminal peptide segment. In eukaryotes, the signal peptide of a nascent precursor protein (pre-protein) directs the ribosome to the rough Endoplasmic Reticulum (ER) membrane and initiates the transport of the growing peptide chain across it. In prokaryotes, the signal peptide directs the pre-protein to the cytoplasmic membrane. However, the signal peptide is not responsible for the final destination of the mature protein; secretory proteins devoid of further address tags in their sequence are by default secreted to the external environment. Signal peptides are cleaved from precursor proteins by an endoplasmic reticulum (ER)-resident signal peptidase or they remain uncleaved and function as a membrane anchor. Signal peptides often have multi-MHC class promiscuity and thus an expanded HLA repertoire. In some embodiments, a fragment of a signal peptide is a fragment capable of binding or being bound by an MHC class molecule. In some embodiments, the MHC molecule is a human leukocyte antigen (HLA).

In some embodiments, the immunogenic peptide comprises a sequence from a vaccine. In some embodiments, the immunogenic peptide comprises a sequence of a fragment of a protein that is in a vaccine. In some embodiments the vaccine is a vaccine suitable for administration to humans. In some embodiments, the vaccine is a human vaccine. In some embodiments, the vaccine is administered to humans. In some embodiments, the vaccine is a vaccine against a pathogen that infects humans. In some embodiments, a pathogen is selected from a bacterium and a virus. In some embodiments, a pathogen is selected from a bacterium, a virus and a parasite. In some embodiments, the vaccine is a tuberculosis vaccine. In some embodiments, the tuberculosis vaccine is the BCG vaccine. In some embodiments, the vaccine is a mumps vaccine. In some embodiments, the vaccine is a measles vaccine. In some embodiments, the vaccine is a diphtheria vaccine. In some embodiments, the vaccine is a SARS-Cov-2 vaccine. In some embodiments, the vaccine is a rabies vaccine. In some embodiments, the vaccine is a hepatitis vaccine. In some embodiments, hepatitis is hepatitis B. In some embodiments, the vaccine is an influenza vaccine. In some embodiments, the influenza is influenza A. In some embodiments, the influenza is influenza B. In some embodiments, the influenza A is H2N2.

In some embodiments, the non-human protein is a tuberculosis protein. In some embodiments, the non-human protein is a mumps protein. In some embodiments, the non-human protein is a herpes simplex virus (HSV) protein. In some embodiments, the non-human protein is a measles protein. In some embodiments, the non-human protein is a diphtheria protein. In some embodiments, the non-human protein is a cytomegalovirus (CMV) protein. In some embodiments, the non-human protein is a severe acute respiratory syndrome coronavirus 2 (SARS-CoV-2) protein. In some embodiments, the non-human protein is a cholera protein. In some embodiments, the non-human protein is a rabies protein. In some embodiments, the non-human protein is a hepatitis B protein. In some embodiments, the non-human protein is an influenza type A protein.

In some embodiments, the non-human protein is selected from a protein provided in Table 2. In some embodiments, the non-human protein is antigen 85B. In some embodiments, the non-human protein is lipoprotein IpqH. In some embodiments, the non-human protein isRv0476/MTO4941. In some embodiments, the non-human protein is BPBP1. In some embodiments, the non-human protein is MT0213. In some embodiments, the non-human protein is tuberculosis protease. In some embodiments, the non-human protein is tuberculosis ATP dependent helicase. In some embodiments, the non-human protein is Rv1334/MT1376. In some embodiments, the non-human protein is lipoprotein lpqV. In some embodiments, the non-human protein is mumps fusion glycoprotein. In some embodiments, the non-human protein is glucoprotein B. In some embodiments, the non-human protein is measles fusion protein. In some embodiments, the non-human protein is diphtheria toxin. In some embodiments, the non-human protein is CMV envelope protein B. In some embodiments, the non-human protein is CMV envelope protein H. In some embodiments, the non-human protein is SARS-CoV-2 spike protein. In some embodiments, the non-human protein is SARS-CoV-2 ORF-7a. In some embodiments, the non-human protein is SARS-CoV-2 ORF-8. In some embodiments, the non-human protein is cholera enterotoxin A. In some embodiments, the non-human protein is cholera outer membrane protein V. In some embodiments, the non-human protein is cholera outer membrane protein W. In some embodiments, the non-human protein is cholera enterotoxin B. In some embodiments, the non-human protein is rabies glycoprotein G. In some embodiments, the non-human protein is hepatitis B external core antigen.

In some embodiments, the immunogenic peptide is selected from SEQ ID NO: 5 and 46-73. In some embodiments, the immunogenic peptide is selected from SEQ ID NO: 5 and 46-73 or a homolog or variant thereof. In some embodiments, a homolog or variant is a homolog or variant that binds or is bound by an MHC class molecule. In some embodiments, the immunogenic peptide is SEQ ID NO: 5.

In some embodiments, the immunogenic peptide comprises at least 10, 11, 12, 13, 14, 15, 16, 17, 18, 19, 20, 21, 22, 23, 24, or 25 amino acids. Each possibility represents a separate embodiment of the invention. In some embodiments, the immunogenic peptide comprises at least 15 amino acids. In some embodiments, the immunogenic peptide comprises at least 20 amino acids. In some embodiments, the immunogenic peptide comprises at least 24 amino acids.

In some embodiments, the second subunit further comprise a second immunogenic peptide. In some embodiments, the second subunit comprises at least 1, 2, 3, 4, 5, 6, 7, 8, 9 or 10 immunogenic peptides. In some embodiments, the second immunogenic peptide is not a signal peptide or a fragment thereof. In some embodiments, the second immunogenic peptide is not a signal peptide. In some embodiments, the first immunogenic peptide is a signal peptide or fragment thereof and any subsequent immunogenic peptide need not be a signal peptide or fragment thereof. In some embodiments, subsequent is C-terminal. In some embodiments, the second immunogenic peptide comprises a sequence from a vaccine suitable for administration to humans. In some embodiments, the second immunogenic peptide is C-terminal to the immunogenic peptide. In some embodiments, the immunogenic peptide is the first immunogenic peptide. In some embodiments, the second immunogenic peptide is selected from SEQ ID NO: 5, 11 and 46-73 or a homolog or variant thereof. In some embodiments, the second immunogenic peptide is selected from SEQ ID NO: 11.

In some embodiments, the second subunit comprises SEQ ID NO: 5 and SEQ ID NO: 11. In some embodiments, SEQ ID NO: 11 is C-terminal to SEQ ID NO: 5. In some embodiments, two immunogenic peptides are separated by a linker. In some embodiments, the first immunogenic peptide and the second immunogenic peptide are separated by a linker. In some embodiments, the second subunit comprises MKRGLTVAVAGAAILVAGLSGCSSGGSGGSGGSNLVPMVATV (SEQ ID NO: 74). In some embodiments, the second subunit consists of SEQ ID NO: 74.

In some embodiments, the chimeric polypeptide further comprises a third subunit. In some embodiments, the third subunit is between the first and second subunits. In some embodiments, the third subunit is C-terminal to the first subunit. In some embodiments, the third subunit is N-terminal to the second subunit. In some embodiments, the third subunit is a linker. In some embodiments, the third subunit is linked to the C-terminus of the Fc. In some embodiments, the third subunit is linked to the C-terminus of the CH3 domain. In some embodiments, the third subunit is at the end of the heavy chain. In some embodiments, the third subunit is at the end of the light chain. In some embodiments, the third subunit is linked to the end of the heavy chain. In some embodiments, the third subunit is linked to the end of the light chain. In some embodiments, the end is the C-terminus. In some embodiments, the end is the N-terminus.

In some embodiments, the third subunit comprises a cleavable moiety. In some embodiments, the third subunit comprises a cleavable linker. In some embodiments, the cleavable moiety is a moiety cleavable in a cell. In some embodiments, the cell is a mammalian cell. In some embodiments, the mammal is a human. In some embodiments, the cleavable moiety is cleavable in the cytoplasm. In some embodiments, the cleavable moiety is cleavable in an endosome. In some embodiments, the cleavable moiety is cleavable in an endosomal vesicle. In some embodiments, the cleavable moiety is cleavable in a lysosome. In some embodiments, the cleavable moiety is cleavable in a lysosomal vesicle. In some embodiments, the cleavable moiety is cleavable in a human.

In some embodiments, the cleavable moiety or linker is a furin-cleavable linker. In some embodiments, a furin-cleavable linker is a furin-sensitive linker. In some embodiments, the third subunit comprises a furin-cleavable sequence. In some embodiments, the canonical furin cleavage sequence comprises RXBR (SEQ ID NO: 45), wherein X is any amino acid and B is any positively charged amino acid. In some embodiments, a positively charged amino acid is selected from R and K. In some embodiments, the furin cleavage sequence comprises RXXR (SEQ ID NO: 75), wherein X is any amino acid. In some embodiments, SEQ ID NO: 75 is RQPR (SEQ ID NO: 76). In some embodiments, the third subunit comprises TRHRQPRGWEQL (SEQ ID NO: 6). In some embodiments, the third subunit consists of SEQ ID NO: 6. In some embodiments, the furin-cleavable linker/moiety comprises SEQ ID NO: 6. In some embodiments, the furin-cleavable linker/moiety consists of SEQ ID NO: 6.

In some embodiments, the first and third subunits are separated by a linker. In some embodiments, the second and third subunits are separated by a linker. In some embodiments, any of the first, second and third subunits are separated by a linker.

In some embodiments, the chimeric polypeptide further comprises an N-terminal signal peptide. In some embodiments, the signal peptide is a leader peptide. In some embodiments, the signal peptide allows for secretion of the chimeric polypeptide. In some embodiments, the signal peptide comprises MKVKVLSLLVPALLVAGAANA (SEQ ID NO: 9). In some embodiments, the signal peptide consists of SEQ ID NO: 9. In some embodiments, the signal peptide is cleaved from the chimeric polypeptide after it is secreted. In some embodiments, the chimeric polypeptide is devoid of an N-terminal signal peptide. In some embodiments, the chimeric polypeptide comprises only 1 signal peptide and it is in the second subunit. In some embodiments, the chimeric polypeptide comprises not more than 2 signal peptides. In some embodiments, the two signal peptides are both in the second subunit. In some embodiments, 1 signal peptide is an N-terminal signal peptide and 1 signal peptide is in the second subunit. In some embodiments, the N-terminal signal peptide is separated from the first subunit by a linker.

In some embodiments, the chimeric polypeptide further comprises a tag. In some embodiments, the tag is an affinity tag. In some embodiments, the tag is a tag usable for affinity purification. In some embodiments, the tag is a C-terminal tag. In some embodiments, the tag is within the chimeric polypeptide. In some embodiments, the tag is a histidine tag. In some embodiments, the tag is a 6x histidine tag. In some embodiments, the tag comprises the sequence HHHHHH (SEQ ID NO: 10). In some embodiments, the tag consists of SEQ ID NO: 10. In some embodiments, the chimeric polypeptide is devoid of a tag. In some embodiments, the tag is separated from the second subunit by a linker.

In some embodiments, the chimeric polypeptide comprises MKVKVLSLLVPALLVAGAANADIQMTQSPSSLSASVGDRVTITCRASQDVNT AVAWYQQKPGKAPKLLIYSASFLYSGVPSRFSGSRSGTDFTLTISSLQPEDFAT YYCQQHYTTPPTFGQGTKVEIKGGGGSGGGGSGGGGSEVQLVESGGGLVQPG GSLRLSCAASGFNIKDTYIHWVRQAPGKGLEWVARIYPTNGYTRYADSVKGR FTISADTSKNTAYLQMNSLRAEDTAVYYCSRWGGDGFYAMDYWGQGTLVT VSSGGGGSGGGGSTRHRQPRGWEQLGGSGGSGGSMKRGLTVAVAGAAILVA GLSGCSSGGSGGSGGSNLVPMVATVHHHHHH (SEQ ID NO: 12) or a homolog or variant thereof. In some embodiments, the chimeric polypeptide consists of SEQ ID NO: 12 or a homolog or variant thereof. In some embodiments, the chimeric polypeptide comprises SEQ ID NO: 12. In some embodiments, the chimeric polypeptide consists of SEQ ID NO: 12.

In some embodiments, the chimeric polypeptide comprises MKVKVLSLLVPALLVAGAANADIQMTQSPSSLSASVGDRVTITCRASQDVNT AVAWYQQKPGKAPKLLIYSASFLYSGVPSRFSGSRSGTDFTLTISSLQPEDFAT YYCQQHYTTPPTFGQGTKVEIKGGGGSGGGGSGGGGSEVQLVESGGGLVQPG GSLRLSCAASGFNIKDTYIHWVRQAPGKGLEWVARIYPTNGYTRYADSVKGR FTISADTSKNTAYLQMNSLRAEDTAVYYCSRWGGDGFYAMDYWGQGTLVT VSSGGGGSGGGGSTRHRQPRGWEQLGGSGGSGGSMKRGLTVAVAGAAILVA GLSGCSSGGSGGSGGSNLVPMVATV (SEQ ID NO: 13) or a homolog or variant thereof. In some embodiments, the chimeric polypeptide consists of SEQ ID NO: 13 or a homolog or variant thereof. In some embodiments, the chimeric polypeptide comprises SEQ ID NO: 13. In some embodiments, the chimeric polypeptide consists of SEQ ID NO: 13.

In some embodiments, the chimeric polypeptide comprises DIQMTQSPSSLSASVGDRVTITCRASQDVNTAVAWYQQKPGKAPKLLIYSASF LYSGVPSRFSGSRSGTDFTLTISSLQPEDFATYYCQQHYTTPPTFGQGTKVEIKG GGGSGGGGSGGGGSEVQLVESGGGLVQPGGSLRLSCAASGFNIKDTYIHWVR QAPGKGLEWVARIYPTNGYTRYADSVKGRFTISADTSKNTAYLQMNSLRAED TAVYYCSRWGGDGFYAMDYWGQGTLVTVSSGGGGSGGGGSTRHRQPRGWE QLGGSGGSGGSMKRGLTVAVAGAAILVAGLSGCSSGGSGGSGGSNLVPMVA TVHHHHHH (SEQ ID NO: 14) or a homolog or variant thereof. In some embodiments, the chimeric polypeptide consists of SEQ ID NO: 14 or a homolog or variant thereof. In some embodiments, the chimeric polypeptide comprises SEQ ID NO: 14. In some embodiments, the chimeric polypeptide consists of SEQ ID NO: 14.

In some embodiments, the chimeric polypeptide comprises DIQMTQSPSSLSASVGDRVTITCRASQDVNTAVAWYQQKPGKAPKLLIYSASF LYSGVPSRFSGSRSGTDFTLTISSLQPEDFATYYCQQHYTTPPTFGQGTKVEIKG GGGSGGGGSGGGGSEVQLVESGGGLVQPGGSLRLSCAASGFNIKDTYIHWVR QAPGKGLEWVARIYPTNGYTRYADSVKGRFTISADTSKNTAYLQMNSLRAED TAVYYCSRWGGDGFYAMDYWGQGTLVTVSSGGGGSGGGGSTRHRQPRGWE QLGGSGGSGGSMKRGLTVAVAGAAILVAGLSGCSSGGSGGSGGSNLVPMVA TV (SEQ ID NO: 15) or a homolog or variant thereof. In some embodiments, the chimeric polypeptide consists of SEQ ID NO: 15 or a homolog or variant thereof. In some embodiments, the chimeric polypeptide comprises SEQ ID NO: 15. In some embodiments, the chimeric polypeptide consists of SEQ ID NO: 15.

In some embodiments, the chimeric polypeptide comprises DIQMTQSPSSLSASVGDRVTITCRASQDVNTAVAWYQQKPGKAPKLLIYSASF LYSGVPSRFSGSRSGTDFTLTISSLQPEDFATYYCQQHYTTPPTFGQGTKVEIKG GGGSGGGGSGGGGSEVQLVESGGGLVQPGGSLRLSCAASGFNIKDTYIHWVR QAPGKGLEWVARIYPTNGYTRYADSVKGRFTISADTSKNTAYLQMNSLRAED TAVYYCSRWGGDGFYAMDYWGQGTLVTVSSGGGGSGGGGSTRHRQPRGWE QLGGSGGSGGSMKRGLTVAVAGAAILVAGLSGCSS (SEQ ID NO: 16) or a homolog or variant thereof. In some embodiments, the chimeric polypeptide consists of SEQ ID NO: 16 or a homolog or variant thereof. In some embodiments, the chimeric polypeptide comprises SEQ ID NO: 16. In some embodiments, the chimeric polypeptide consists of SEQ ID NO: 16.

In some embodiments, the chimeric polypeptide comprises an amino acid sequence selected from SEQ ID NO: 12-16. In some embodiments, the chimeric polypeptide comprises an amino acid sequence selected from SEQ ID NO: 12-16 or a homolog or variant thereof. In some embodiments, the chimeric polypeptide consists of an amino acid sequence selected from SEQ ID NO: 12-16. In some embodiments, the chimeric polypeptide consists of an amino acid sequence selected from SEQ ID NO: 12-16 or a homolog or variant thereof.

In some embodiments, the chimeric polypeptide comprises an amino acid sequence selected from SEQ ID NO: 14-16. In some embodiments, the chimeric polypeptide comprises an amino acid sequence selected from SEQ ID NO: 14-16 or a homolog or variant thereof. In some embodiments, the chimeric polypeptide consists of an amino acid sequence selected from SEQ ID NO: 14-16. In some embodiments, the chimeric polypeptide consists of an amino acid sequence selected from SEQ ID NO: 14-16 or a homolog or variant thereof.

In some embodiments, a homolog comprises at least 90% homology. In some embodiments, a homolog comprises at least 92% homology. In some embodiments, a homolog comprises at least 95% homology. In some embodiments, a homolog comprises at least 97% homology. In some embodiments, a homolog comprises at least 99% homology. In some embodiments, the homolog or variant is capable of binding to the cancer antigen. In some embodiments, the homolog or variant is capable of binding the cancer antigen on a surface of a target cell. In some embodiments, the target cell is a cancer cell. In some embodiments, the homolog or variant is capable of inducing expression of the immunogenic peptide on a surface of the target cell. In some embodiments, the homolog or variant is capable of binding the cancer antigen on the target cell surface and expressing the immunogenic peptide on the target cell surface.

According to another aspect, there is provided a nucleic acid molecule comprising a nucleotide sequence encoding a chimeric polypeptide of the invention. The terms “polynucleotide,” “polynucleotide sequence,” “nucleic acid sequence,” and “nucleic acid molecule” are used interchangeably herein. These terms encompass nucleotide sequences and the like. A polynucleotide may be a polymer of RNA or DNA that is single- or double-stranded, that optionally contains synthetic, non-natural or altered nucleotide bases. In some embodiments, the nucleic acid molecule is DNA. In some embodiments, the nucleic acid molecule is RNA. In some embodiments, the nucleic acid molecule is a plasmid. In some embodiments, the nucleic acid molecule is an expression vector.

In some embodiments, the nucleic acid molecule further comprises at least one regulatory element. In some embodiments, the at least one regulatory element is operatively linked to the nucleotide sequence encoding the chimeric polypeptide. In some embodiments, the at least one regulatory element is capable of driving expression of the nucleotide sequence. In some embodiments, driving expression is expression in a target cell. In some embodiments, the at least one regulatory element is capable of expressing the nucleotide sequence. In some embodiments, the target cell is a human cell. In some embodiments, the target cell is a cancer cell. In some embodiments, the target cell is a packaging cell. In some embodiments, the target cell is a bacterial cell. In some embodiments, the bacterial cell is an E. coli cell. In some embodiments, the target cell is an expression cell. In some embodiments, the target cell is a furin insensitive cell. In some embodiments, the target cell is a furin sensitive cell. In some embodiments, the target cell does not express furin. In some embodiments, the target cell expresses furin.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene within a cell is well known to one skilled in the art. It can be carried out by, among many methods, transfection, viral infection, or direct alteration of the cell's genome. In some embodiments, the nucleotide sequence is in an expression vector such as plasmid or viral vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene is operably linked to a promoter. In some embodiments, the regulatory element is a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

A person with skill in the art will appreciate that a gene can also be expressed from a nucleic acid construct administered to the individual employing any suitable mode of administration, described hereinabove (i.e., in vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex vivo gene therapy).

By another aspect, there is provided a cell comprising a nucleic acid molecule of the invention. In some embodiments, the cell is a host cell.

“Cancer” and “cancerous” refer to or describe the physiological condition in mammals that is typically characterized by unregulated cell growth. Examples of cancer include, but are not limited to hematopoietic cancers (e.g., lymphomas and leukemias), brain cancer, breast cancer, mesothelioma cancer, colon cancer, lung cancer, hepatocellular cancer, gastric cancer, pancreatic cancer, cervical cancer, ovarian cancer, liver cancer, bladder cancer, cancer of the urinary tract, thyroid cancer, renal cancer, head and neck cancer, testicular cancer, skin cancer and prostate cancer, as well as the various metastases of these cancers.

“Immune cell” as used herein refers to the cells of the mammalian immune system including but not limited to antigen presenting cells, B-cells, basophils, cytotoxic T-cells, dendritic cells, eosinophils, granulocytes, helper T-cells, leukocytes, lymphocytes, macrophages, mast cells, memory cells, monocytes, natural killer cells, neutrophils, phagocytes, plasma cells and T-cells.

“Immune response” as used herein refers to immunities including but not limited to innate immunity, humoral immunity, cellular immunity, immunity, inflammatory response, acquired (adaptive) immunity, autoimmunity and/or overactive immunity.

“Polynucleotide” as used herein includes but is not limited to DNA, RNA, cDNA (complementary DNA), mRNA (messenger RNA), RNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA.

“Single chain variable fragment”, “single-chain antibody variable fragments” or “scFv” antibodies as used herein refer to forms of antibodies comprising the variable regions of only the heavy and light chains, connected by a linker peptide.

The terms “T-cell” and “T-lymphocyte” are interchangeable and used synonymously herein. Examples include but are not limited to naive T cells, central memory T cells, effector memory T cells or combinations thereof.

In some embodiments, the cancer is a tumor. “Tumor,” as used herein refers to all neoplastic cell growth and proliferation, whether malignant or benign, and all pre-cancerous and cancerous cells and tissues. In some embodiments, the tumor is a metastasis.

A “subunit” of a fusion polypeptide disclosed herein is defined as a stretch of amino acids of the polypeptide, which stretch defines a unique functional unit of said polypeptide such as provides binding motif towards a target.

### Pharmaceutical Compositions

By another aspect, there is provided a pharmaceutical composition comprising a chimeric polypeptide of the invention.

According to certain embodiments, the chimeric polypeptide of the present invention is administered in the form of a pharmaceutical composition. As used herein, the term “pharmaceutical composition” refers to a preparation of the chimeric polypeptide with other chemical components such as pharmaceutically acceptable carriers and excipients. The purpose of a pharmaceutical composition is to facilitate administration of an active ingredient to an organism and enhance its stability and turnover.

In some embodiments, the pharmaceutical composition further comprises a pharmaceutically acceptable carrier, excipient or adjuvant.

In some embodiments, the pharmaceutical composition comprises a therapeutically effective amount of a chimeric polypeptide of the invention. An “effective amount” is an amount sufficient to effect beneficial or desired results. An effective amount can be administered in one or more administrations.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

The term “pharmaceutically acceptable” means approved by a regulatory agency of the Federal or a state government or listed in the U. S. Pharmacopeia or other generally recognized pharmacopeia for use in animals, and more particularly in humans.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

The pharmaceutical compositions of the present invention can be manufactured by processes well known in the art, e.g., by means of conventional mixing, dissolving, granulating, dragee-making, levigating, emulsifying, encapsulating, entrapping, spray drying or lyophilizing processes.

According to certain exemplary embodiments, pharmaceutical compositions, which contain the chimeric polypeptide as an active ingredient, are prepared as injectable, either as liquid solutions or suspensions, however, solid forms, which can be suspended or solubilized prior to injection, can also be prepared. According to yet additional embodiments, the chimeric polypeptide-containing pharmaceutical composition is formulated in a form suitable for subcutaneous administration.

Methods of introduction of a pharmaceutical composition comprising the chimeric polypeptide include, but are not limited to, intravenous, subcutaneous, intramuscular, intraperitoneal, oral, topical, intradermal, transdermal, intranasal, epidural, ophthalmic, vaginal and rectal routes. The pharmaceutical compositions can be administered by any convenient route, for example by infusion or bolus injection, by absorption through epithelial linings (e.g., oral mucosa, rectal and intestinal mucosa, etc.), and may be administered together with other therapeutically active agents. The administration may be localized, or may be systemic.

In some embodiments, the pharmaceutical composition is formulated for systemic administration. pharmaceutical composition is formulated for intratumoral administration.

As used herein, the terms “administering,” “administration,” and like terms refer to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, or intraperitoneal.

The dosage administered will be dependent upon the age, health, and weight of the recipient, kind of concurrent treatment, if any, frequency of treatment, and the nature of the effect desired.

Alternatively, the active ingredient may be in powder form for constitution with a suitable vehicle, e.g., a sterile, pyrogen-free, water-based solution, before use.

### Methods of Use

By another aspect, there is provided a method of expressing an immunogenic peptide on a surface of a target cell, the method comprising contacting the target cell with a chimeric polypeptide of the invention, or a pharmaceutical composition of the invention, thereby expressing the immunogenic peptide on a surface of a target cell.

By another aspect, there is provided a method of preventing, ameliorating or treating cancer in a subject in need thereof, the method comprising administering to the subject a chimeric polypeptide of the invention, or a pharmaceutical composition of the invention, thereby preventing, ameliorating or treating cancer in a subject.

In some embodiments, the target cell is a cancer cell. In some embodiments, the target cell is mammalian cell. In some embodiments, the target cell expresses the cancer antigen. In some embodiments, the target cell expresses the cancer antigen on its surface. In some embodiments, the target cell comprises an agent that cleaves the cleavable moiety. In some embodiments, the agent is an enzyme. In some embodiments, the enzyme is a peptidase. In some embodiments, the agent is a

In some embodiments, the cancer is a cancer that expresses the cancer antigen. In some embodiments, the cancer is a cancer that expresses the cancer antigen on a surface of a cell of the cancer. In some embodiments, at least 10, 15, 20, 25, 30, 35, 40, 45, 50, 55, 60, 65, 70, 75, 80, 85, 90, 95 or 100% of the cancer cells expresses the cancer antigen. Each possibility is a separate embodiment of the invention. In some embodiments, express is expresses on the cell surface. In some embodiments, at least 20% of the cancer cells expresses the cancer antigen. In some embodiments, at least 30% of the cancer cells expresses the cancer antigen. In some embodiments, at least 40% of the cancer cells expresses the cancer antigen. In some embodiments, at least 50% of the cancer cells expresses the cancer antigen. In some embodiments, the cancer is a HER positive cancer. In some embodiments, the cancer is positive for the cancer antigen.

A “subject” is a vertebrate, preferably a mammal, more preferably a human. The term “mammal” is used herein to refer to any animal classified as a mammal, including, without limitation, humans, domestic and farm animals, and zoo, sports, or pet animals, such as sheep, dogs, horses, cats, cows, rats, pigs, apes such as cynomolgus monkeys and etc., to name only a few illustrative examples. Preferably, the mammal herein is human. In some embodiments, the subject is a human. In some embodiments, the subject suffers from cancer. In some embodiments, the subject has already been vaccinated. In some embodiments, the subject has already received a vaccine. In some embodiments, the vaccine comprises an amino acid sequence present in the second subunit. In some embodiments, the vaccine comprises an amino acid sequence present in the immunogenic peptide. The subject is vaccinated before the administering. In some embodiments, vaccinated is receiving a vaccine.

In some embodiment, the method further comprises administering a vaccine to the subject. In some embodiments, the vaccine is administered before administering the chimeric polypeptide or pharmaceutical composition. In some embodiments, the vaccine is administered at the same time as administering the chimeric polypeptide or pharmaceutical composition.

In some embodiments, the vaccine is a tuberculosis vaccine and the second subunit comprises a tuberculosis peptide. In some embodiments, the tuberculosis vaccine is the BCG vaccine and the second subunit comprises an amino acid sequence selected from SEQ ID NO: 5 and 46-53. In some embodiments, the second subunit comprises SEQ ID NO: 5.

In some embodiments, the method further comprises treating the subject with an immunotherapy. In some embodiments, the immunotherapy is adoptive T cell transfer. In some embodiments, the T cells are tumor infiltrative lymphocytes (TILs). In some embodiments, the immunotherapy is chimeric antigen receptor (CAR therapy). In some embodiments, the CAR therapy is CAR-T cell therapy. In some embodiments, the CAR therapy is CAR-natural killer (NK) cell therapy. In some embodiments, the adoptive T cell is a CAR-T. In some embodiments, the T cell is specific to a sequence present in the second subunit. In some embodiments, the T cell is specific to a sequence present in the immunogenic peptide. In some embodiments, the CAR is specific to a sequence present in the second subunit. In some embodiments, the CAR is specific to a sequence present in the immunogenic peptide.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Material and Methods

Peptides: The Her2Neu and BCG-derived peptides were chemically synthesized (Genemed Synthesis Inc., San Antonio, TX, USA) by fully automated, solid-phase, peptide synthesis using the fluorenylmethyloxycarbonyl (Fmoc)/tBu-strategy and Rink-amide-polystyrene resin. The purity and identity of the peptide was determined by HPLC-Mass Spectra analysis and was >95% for all peptides.

Designing and constructing the Her2-ScFv and Her2-ScFv-BCG-CMV Chimeras: The design and construction of the chimera used standard, E-coli, molecule biology methods.

ELISA for evaluating Her2-ScFv and Her2-ScFv-BCG-CMV (chimera) batch to batch comparison: ELISA plates (F96 Maxisorp, Nunc, Denmark) were activated for 1 hr with 0.1% glutaraldehyde (Sigma, Israel) in carbonate buffer, pH=9. Next, plates were coated with 50 ul of Her2 peptide (5 ug/ml), in carbonate buffer (ON, 4° C.), followed by blocking (2 hrs, RT) with PBS supplemented with 5% FBS and 0.04% Tween 20 (ICN Biomedical Inc, USA) (blocking buffer). Chimera samples were diluted 1:1 in the blocking buffer for 6 serial dilutions. Positive control Human anti-Her2 antibody was used at a starting concentration of 100 ug/ml with an additional x6 double dilutions. Samples of 50 ul were added to the ELISA plate and incubated for 2 hrs, at RT and then washed x6 times with PBS supplemented with 0.04% tween-20 (washing solution). Next the secondary antibody, HRP-conjugated anti-Human IgG (Jackson ImmunoResearch, USA); 50 ul/well, was added for 1 hr to the ELISA plate with the Human anti-Her2 Herceptin at a working dilution of 1:10,000 in blocking buffer. For samples analysis, anti-His HRP (Biolegend cat #63260) 50 ul/well, was added at a working dilution of 1:100 in blocking buffer for 1 hr to the ELISA plate with the evaluated chimera samples. After extensive washing, plates were developed with TMB/E solution (CHEMICON, Millipore, USA) according to the manufacturer's instructions.

Internalization of the Her2-ScFv-BCG-CMV chimera into tumor cell lines: Tumor cell preparations: 3 ml of cell suspension were plated at the final concentration of 0.5-1×106 cell/ml into several 6-well plates (CellStar Greiner cat #657160). One plate (6 wells) was used for every evaluated cell-line.

Internalization: 50 ug/ml of the Her2-ScFv-BCG-CMV chimera were added in each well of the 6-well plates. Separates 6-well plates were used for each evaluated doses of the chimera. The culture were placed at 37° C. in a 5% CO₂ tissue culture incubator. Cells from 2 wells of each kind of culture conditions were collected after 6, 24, 48 and 72 hrs and stained for membranal and intracellular localization of the Her2-ScFv-BCG-CMV chimera. Detection of both membranal or intracellular localization of the chimera was done by FACS analysis using staining with anti-Hu secondary antibody (Biolegend cat #109-136-088) or anti His-tag antibody (SantoCrus cat #AF 547). Baseline internalization levels are analyzed as described, on intact cells at day zero, after 1 hr exposure at RT with 50 ug/ml of chimera.

For intracellular stating (ICS), cells were detached by Trypsin and washed with Glycine buffer (pH 3) (Home preparation) for 0.5 min followed by washing with cold PBS (for removing membrane binding of the Chimera). Next, cells underwent fixation for 20 min and then permeabilization with the Leucoperm kit (Bio-Rad cat #Buf 09149239) Next, cells were stained with secondary Ab and washed once with Glycine buffer and an additional x2 times with PBS supplemented with 3% FCS+0.1 sodium azide (FACS buffer).

T-cell specific Cytotoxicity of targets mediated by the Her2-ScFv-BCG-CMV Chimera: Preparation of peptide-pulsed dendritic cells: Dendritic Cells (DCs) were enriched from blood samples obtained from naïve donors. Briefly, PBMC were separated with Ficoll (Sigma 1077-100 ml, Israel) at 2,400 rpm for 30 min and cultured at a concentration of 2.5×10\hat{ }6/ml in complete RPMI-1640 medium for 4 hrs at 37° C. in tissue culture 150 mm×25 mm dishes (CellStar, Greiner, Germany). Adherent cells were collected and re-cultured in serum-free DCCM-1 medium (Biological Industries, Beit Haemek, Israel) supplemented with L-Glutamine, human IL-4 (1000 IU/ml) and GM-CSF 80 ng/ml. Cultures at the concentration of 1×10\hat{ }6 cells/ml were performed in 6-well tissue culture plates (Costar, Corning, Germany) for 7 days at 37° C. On day 7, floating cells were collected, washed with PBS, and loaded with 50 μg/ml of specified peptide for 18 hrs at 37° C. DC-loaded cells were then utilized for the different immunological assays.

Effector Cells Preparations: peripheral blood mononuclear cell (PBMC) isolated from naïve donors (Israel's national blood bank) were separated by Ficoll gradient (Sigma Histopaqe), washed x3 times with PBS and divided to three separate tubes. Two tubes were kept frozen at 80° C., while a third tube of PBMC was counted and re-suspended at a final concentration of 4×10\hat{ }6/ml in complete RPMI-1640 medium (Bet Ha Emek cat #01-104-1A), transferred to 150 mm×25 mm plates (Celstar Grainer Germany) and incubated at 37° C. for 4 hrs. On day 6, DC were collected and loaded with 50 ug/ml of the stimulated peptide for ON. Next, one tube of frozen PBMC was thawed and mixed with peptide loaded DC for a 1st stimulation at a ratio of 20:1 for 7 days in RPMI-1640 complete medium supplemented with 100 IU/ml Hu-IL7. For the 2^{nd} stimulation, PBMC was transferred to peptide-loaded monocytes for an additional 7 days of culturing. At that point, T cells were collected, washed and re-suspended in RPMI-1640 medium supplemented with 5% Hu ab serum, 2 ug/ml of stimulated peptide and 100u/ml of Hu IL-2 for a 3^{rd} stimulation of 48 hrs. At the end of the 3^{rd} stimulation, T cells were used as effector cells.

Target cells Preparation: OvCar-3 human ovarian carcinoma cell line was used as target cells. Cells were cultured with 50 ug/ml of Chimera for 24, 48 and 72 hrs. Cells cultured w/o Chimera were used as a negative control. On the day of the experiment, cells were detached from cultured pales with EDTA solution, washed and labeled with DiOC according to the kit manufacturer's protocol (Life/Dead Cell-mediated Cytotoxicity Kit L7010). Next, cells were mixed with effector cells at a ratio of 20:1 effector cells to target cells for 2 hrs and cultured at 37° C. At the end of this incubation cells were stained with Propidium Iodide and evaluated by FACS analysis. Specific killing was calculated as follows: (% of killing Her2scFv-BCG-CMV/% of killing Her2scFv)*100. This ratio of effector cells to target cells was used for all experiments unless stated otherwise.

### Example 1: Design and Construction of the Chimera

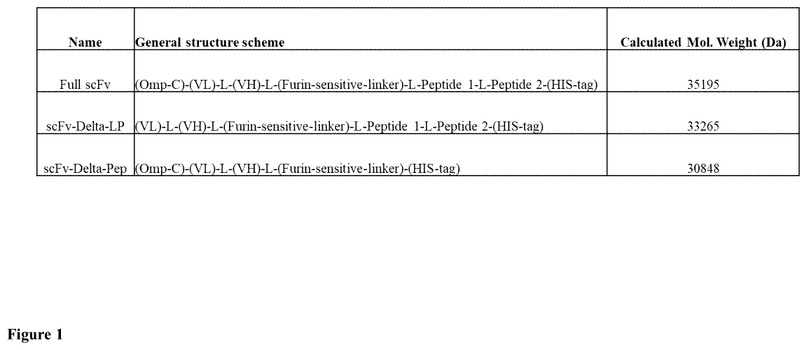

Three scFv chimeric molecules were designed and the constructs are presented schematically in FIG. 1. These chimeric molecules function by binding to a cancer specific antigen, which results in cell internalization, followed by endosomal escape of an immunogenic peptide which is then presented on the tumor cell's surface via MHC-I molecules thus making the tumor visible to the subject's immune system. A single chain antibody (scFv) against a cancer associated antigen is used as a targeting moiety to bring the chimera to the tumor and cause internalization. Bacterial and/or viral signal peptides (SPs) are used as the immunogenic peptide. Use of SPs has a double benefit, firstly they are highly immunogenic and can cause a robust immune response; secondly, they are recognized by the cells own transport system and are automatically displayed by MHC-I molecules without any further modification or intervention. Thus, the use of SPs ensures surface expression of the foreign immunogenic sequence even in TAP-deficient tumors. Other viral/bacterial sequences may also be included downstream of the SP as the SP will ensure surface delivery. These other sequences may be sequences from common infectious agents ensuring the subject already has immunity to them or can be sequences targeted by an adjuvant vaccine that can be administered concomitantly with the chimera. Lastly, a cleavable linker is used to separate the targeting moiety and the immunogenic peptide to ensure endosomal escape of the immunogenic peptide.

An anti-Her2 scFv was selected for the targeting moiety of the chimeric molecules. HER2/neu is a member of the human epidermal growth factor receptor family. Amplification or overexpression of this oncogene have been shown to play an important role in the development and progression of a variety of tumors, including certain aggressive types of breast cancer. HER2/neu has been shown to be highly differentially expressed on tumor cells with much higher cell-surface density compared to healthy tissue. It is thus, an ideal target molecule for this proof-of-concept experiment, though, clearly other anti-cancer scFvs could have been selected.

The scFv contains two variable regions (VL and VH) separated by a (GGGGS) 3 linker. The first variable region consists of the sequence DIQMTQSPSSLSASVGDRVTITCRASQDVNTAVAWYQQKPGKAPKLLIYSASF LYSGVPSRFSGSRSGTDFTLTISSLQPEDFATYYCQQHYTTPPTFGQGTKVEIK (SEQ ID NO: 1). The second variable region consists of the sequence EVOLVESGGGLVQPGGSLRLSCAASGFNIKDTYIHWVRQAPGKGLEWVARIY PTNGYTRYADSVKGRFTISADTSKNTAYLQMNSLRAEDTAVYYCSRWGGDG FYAMDYWGQGTLVTVSS (SEQ ID NO: 2). With the (GGGGS) 3 linker (SEQ ID NO: 3), the full sequence of the anti-Her2 scFv is provided in SEQ ID NO: 4.

The Bacillus Calmette-Guérin (BCG) anti-tuberculosis vaccine is the most commonly used live tuberculosis vaccine currently being employed. A highly immunogenic, pan-HLA SP derived from BCG was previously identified (Kovjazin et al., 2013, “Characterization of novel multiantigenic vaccine candidates with pan-HLA coverage against Mycobacterium tuberculosis”, Clin. Vaccine Immuno., March; 20(3):328-40, herein incorporated by reference in its entirety) and employed in this chimera. The sequence of the BCG-derived SP is MKRGLTVAVAGAAILVAGLSGCSS (SEQ ID NO: 5).

For the purposes of endosomal escape and release of the immunogenic peptide from the scFv a cleavable linker was inserted between the C-terminus of the scFv and the N-terminus of the SP. In this instance the cleavable linker was a furin-sensitive linker consisting of the sequence TRHRQPRGWEQL (SEQ ID NO: 6). Furin is highly expressed in the Golgi and endosomal pathways and thus this linker ensures that the immunogenic peptide is separated from the targeting moiety.

The various domains of the chimera (targeting, immunogenic, cleavable) were separated by glycine-serine linkers. For example, between the scFv and the furin-sensitive linker there was a GGGGSGGGGS (SEQ ID NO: 7) linker and between the furin-sensitive linker and the SP there was a GGSGGSGGS (SEQ ID NO: 8) linker.

Three other optional domains were included in the full-length chimeric molecule. The first is a leader peptide for secretion of the chimeric molecule. The N-terminal leader peptide (or signal peptide) selected was from Omp-C and consisted of the sequence MKVKVLSLLVPALLVAGAANA (SEQ ID NO: 9). Of course, any suitable SP could have been used to ensure secretion of the chimera. A C-terminal His-tag, consisting of HHHHHH (SEQ ID NO: 10) was used for purification of the chimeric molecule, but is not essential for the molecules function. Lastly, a second immunogenic peptide was inserted C-terminally to the BCG-derived SP. A 9-mer from cytomegalovirus (CMV) was used as the second immunogenic peptide; it consisted of the sequence NLVPMVATV (SEQ ID NO: 11). It will be understood however, that any immunogenic peptide that will enhance tumor targeting by a subject's immune cells can be selected for the second peptide. As there is no cleavage site between the SP and the second peptide, they will be transferred together. The SP ensures delivery into the lumen of the ER. This facilitates HLA-I mediated surface expression and so the sequence of the second peptide can be selected based on the subject's known immunity or based on an adjuvant vaccine therapy to be given to the subject. Optional glycine-serine linkers can be inserted between any of these sections. In the full chimeric molecule, a GGSGGSGGS (SEQ ID 8) is between the SP and the second immunogenic peptide.

The final full chimeric molecule is therefore represented by SEQ ID NO: 12 and consists of the sequence MKVKVLSLLVPALLVAGAANADIQMTQSPSSLSASVGDRVTITCRASQDVNT AVAWYQQKPGKAPKLLIYSASFLYSGVPSRFSGSRSGTDFTLTISSLQPEDFAT YYCQQHYTTPPTFGQGTKVEIKGGGGSGGGGSGGGGSEVQLVESGGGLVQPG GSLRLSCAASGFNIKDTYIHWVRQAPGKGLEWVARIYPTNGYTRYADSVKGR FTISADTSKNTAYLQMNSLRAEDTAVYYCSRWGGDGFYAMDYWGQGTLVT VSSGGGGSGGGGSTRHRQPRGWEQLGGSGGSGGSMKRGLTVAVAGAAILVA GLSGCSSGGSGGSGGSNLVPMVATVHHHHHH. This molecule has a predicted molecular weight of 35195.27 kDa. It will be understood however that as the His-tag is not needed for function the full chimera can also be considered SEQ ID NO: 13. Similarly, the N-terminal leader peptide is removed upon secretion and so the full chimeric molecule can also be considered SEQ ID NO: 14 which has the His-tag or SEQ ID NO: 15 which lacks the His-tag. Indeed, the full chimeric molecule does not even need the second peptide and so the core molecule can be viewed as SEQ ID NO: 16.

For the purposes of evaluating chimera production two other derivative molecules were generated (see FIG. 1). The first is identical to the full chimera but lacks the leader peptide which would be cleaved off during secretion (scFv-Delta-LP). The second has removed both immunogenic peptides and the furin-sensitive linker is followed by the His-tag (scFv-Delta-Pep).

With the sequence of the chimeric molecule in hand, the nucleic acid sequence encoding the chimera was codon optimized for expression in E. coli. The codon-optimized nucleic acids sequence encoding the full-length chimera consisted of SEQ ID NO: 17. The nuclscFv-Delta-LP and scFv-Delta-Pep constructs were based on this sequence, but with the nucleotides encoding the deleted sections removed.

### Example 2: Screening Test for Expression of scFv in Various E. coli Strains E. coli BL21

For routine protein expression, BL21 is an ideal starting point strain. The strain was first commercialized in 1990 and has remained the gold standard among expression hosts ever since. The expression of the three scFv variants was tested and partially optimized in a small-scale volume. It can be difficult to express proteins containing internal signal peptides in a heterologous system. Signal peptides are highly hydrophobic and adversely affect the stability, translation and folding of the protein. Preliminary experimentation to produce molecules with multiple signal peptides found that it was difficult and poor yields, or no yield at all, was often the result. As the chimeric molecules have several signal peptides the method of protein expression was carefully analyzed.

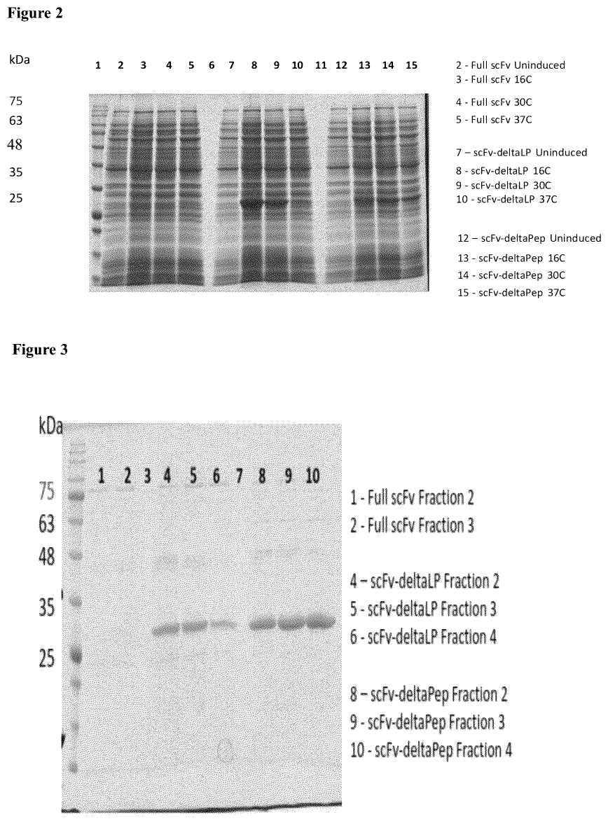

The effect of temperature on protein folding has been well documented and is one of the most common factors to be optimized during production of heterologous proteins. Therefore, the role of temperature on scFv expression was investigated by growing the BL21 cells initially at 37° C. until the cells reached mid-log growth phase. For this purpose, BL21 cells were transformed with pET-21 plasmids harboring the appropriate genes (pET21-Full-scFv, pET21-deltaLP and pET21-scFv-deltaPep). Growing medium of about 200 ml was used to grow the cells up to a 0.8 OD and 30 ml aliquots of un-induced cell samples were withdrawn, centrifuged and the pellets were stored at −20° C. The entire cell suspension was divided into 3 flask and cell suspension samples were induced by IPTG at a final concentration of 0.1 mM. The induced samples were incubated separately under different incubation temperatures and periods (16° C.-24 hr, 30° C.-4 hr and 37° C.-3 hr). Following the induction cells were centrifuged, pellets of the induced and uninduced cells were resuspended in TBS and lysed by sonication. Portions of the lysates were analyzed by SDS-PAGE on a 12% gel. The results showing general expression levels under the different induction temperatures are depicted in FIG. 2.

SDS-PAGE of the soluble fraction samples after the different induction temperature conditions shows an indicative presence of bands that correspond to the expected molecular weight of the scFv-delta-LP and sc-Fv-delta Pep. No visible induction is visible for the full scFv under the tested expression conditions. The expression levels for scFv-deltaLP and sc-Fv-deltaPep are greatly prone to the fermentation/induction temperature. Generally, the level of the expression was inversely proportional to induction temperature, resulting in high expression at 16° C. and low expression at 37° C. This applies to both the scFv variants.

To confirm the SDS-PAGE results, pooled samples (16° C., 30° C. and 37° C. for each variant) were purified by nickel chelate affinity chromatography. The loading volume for each variant was about 15 ml soluble lysate and the nickel beads volume was ˜0.3 ml. The scFv variants were eluted under high imidazole concentration (200 mM) and 2 or 3 relevant elution fractions were analyzed by SDS-PAGE. FIG. 3 summarizes the results of the affinity purification process.

The presence of specific bands that have been eluted by high imidazole concentration and that correspond to scFv-deltaLP and scFv-deltaPep were evident over the various eluted fractions. The molecular weight of the bands was in agreement with the theoretical molecular weight of these molecules at ˜30 kDa.

Generally, the use of Origami™ 2 host strains or E. coli Shuffle did not improve the overall expression performances of the two variants that were efficiently expressed in the BL21 strain. Neither increased the expression level of the full scFv either.

### Example 3: Screening Test for Expression of scFv in Various E. coli Strains—the Effect of IPTG

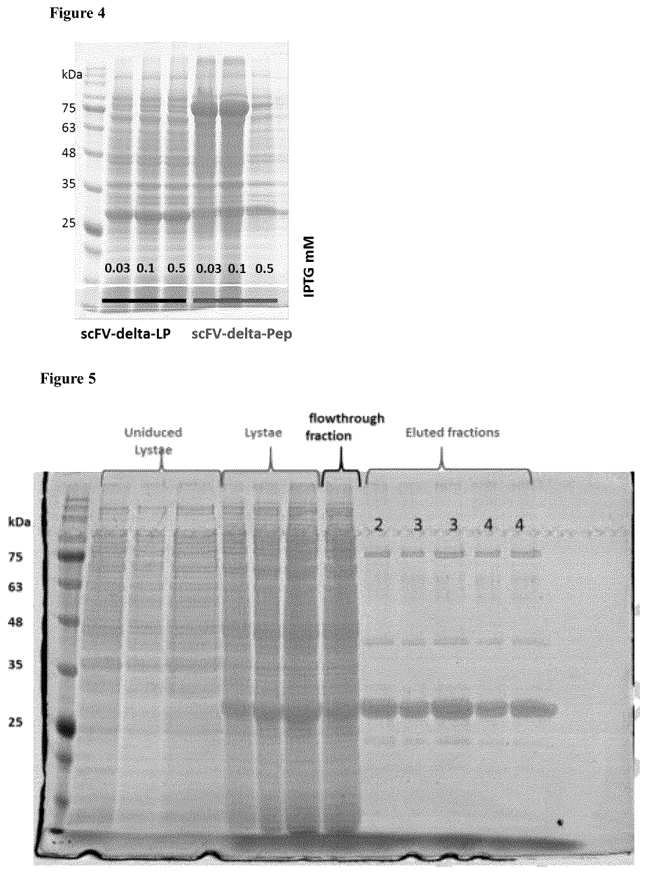

The BL21 strain that showed reasonable levels of expression of scFV-Delta-LP and scFv-delta Pep was preferred for the next optimization step. For this purpose, BL21 cells were transformed by pET-21 plasmids harboring the appropriate constructs (pET21-delta-LP and pET21-scFv-delta-Pep). Growing medium of about 150 ml was used to grow the cells up to 0.8 OD and aliquot of 20 ml of un-induced cells samples were withdrawn, centrifuged and the pellets were stored at −20° C. The entire cell suspension was divided into 3 and cell suspension samples were induced with 0.03 mM, 0.1 mM or 0.5 mM IPTG. The induced samples were incubated at 16° C. overnight. The expression of the three scFv variants is depicted in FIG. 4.

The cell pellet that was generated during the fermentation was resuspended in ice-cold PBS plus protease inhibitors cocktail. The resuspended pellet was immediately sonicated in ice-cold bath, centrifuged and 4° C. and affinity purified. A rough estimation shows that under the fermentation and induction conditions one can reach an amount of 5-7 mg of purified scFv-delta-LP per 1 liter growing medium. Samples taken after each step including the affinity purification were analyzed by SDS-PAGE (FIG. 5).

### Example 4: Purification Under the Optimized Conditions

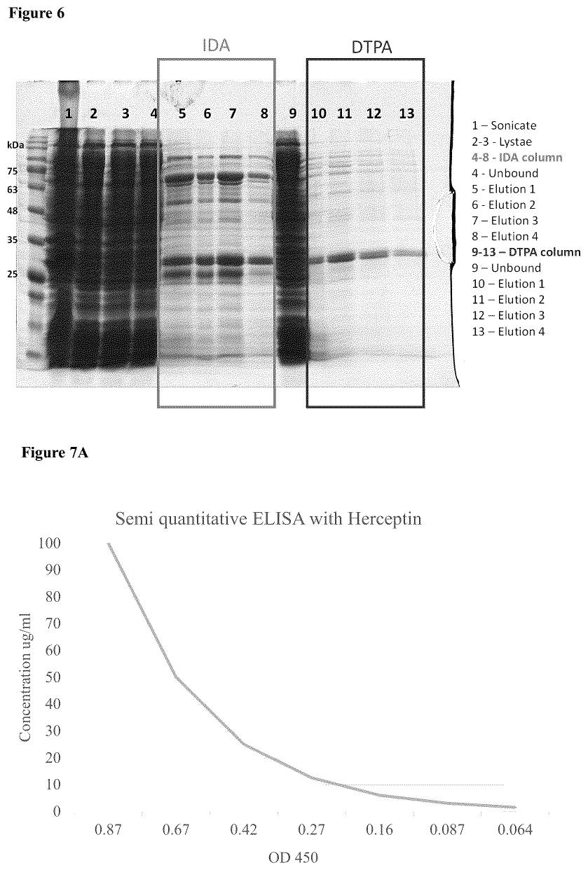

Overnight induced cells were centrifuged, a cell pellet was collected, re-suspended in PBS supplemented with Protease Inhibitor cocktail and lysed by sonication. The lysed sample was centrifuged and affinity purified on two types of nickel-chelate resin (commercial IDA-resin and DTPA-resin). The purification results are summarized in FIG. 6.

Affinity purification of scFv-delta-PEP on IDA resin resulted in a significant enrichment of the band that corresponds with the molecular weight of the construct (30 Kda) and indicates high expression of this construct under the fermentation conditions. Affinity purification of the same sample on DTPA resulted in a much higher level of purification though also an increased loss of product. Thus, purification of the both the test peptide scFv-delta LP and the control peptide scFV-delta-PEP was possible. A purity of greater than 95% was achieved. Thus, the Delta-LP construct was selected as the therapeutic molecule for further testing, as the absence of the leader peptide in the final therapeutic molecule is irrelevant and the Delta-Pep construct was used as the negative control as it lacked any immunogenic sequence.

### ELISA:

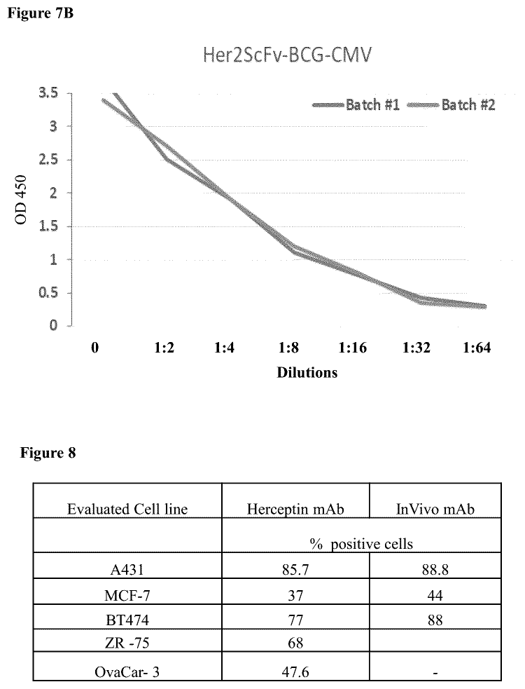

To confirm the Her2/Neu specificity and to quantify the Her2-ScFv and Her2-ScFv-BCG-CMV chimeras, an ELISA assay was established. Since the Her2/Neu binding portion of the chimera is based on the therapeutic agent Herceptin, the Herceptin epitope (synthetic peptide) was used as the primary antigen in this assay. HRP conjugated anti-human or anti-his tag antibodies were used as the detecting antibodies. As can be seen in FIG. 7A, serial dilutions of commercially available anti-Her2/Neu Herceptin antibody gave an OD curve that can be semi-quantitatively used to estimate the amount of unknown quantities of any evaluated chimera having the same Her2/Neu specificity. Importantly, as can be seen in FIG. 7B, different Her2-ScFv-BCG-CMV batches gave a similar Her2/Neu-specificity. Overall, 5 different batchs of Her2-ScFv and Her2-ScFv-BCG-CMV were validated at several time points and temperatures post-production for antigen specificity and chimera stability using this method. All 5 batches were found to be comparable. Further, all subsequently produced batches were also examined and found to be comparable.

### Example 5: Her2-ScFv-BCG-CMV Functionality and Internalization Analysis

In order to be functional, the Her2-ScFv-BCG-CMV must be internalized into Her2/Neu positive tumor cells. This is not trivial. An ScFv does not have the ability to induce receptor cross linking like a full antibody. Moreover, its antigenic payload could interfere with the internalization process. For these reasons, the ability of the Her2-ScFv-BCG-CMV chimera to internalise into a number of Her2/Neu positive cell lines was evaluated. First, Her2/Neu expression was confirmed using FACS analysis. As can be seen in FIG. 8, the A431, BT474, ZR-75 and OvCar-3 cell lines all have a moderate to high Her2/Neu expression as assessed by two independent anti-Her2/Neu mAb.

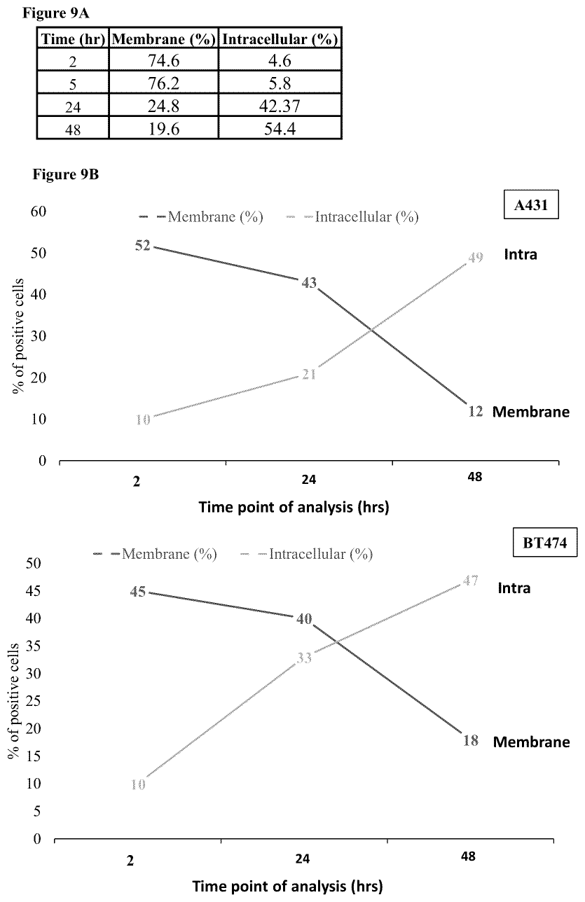

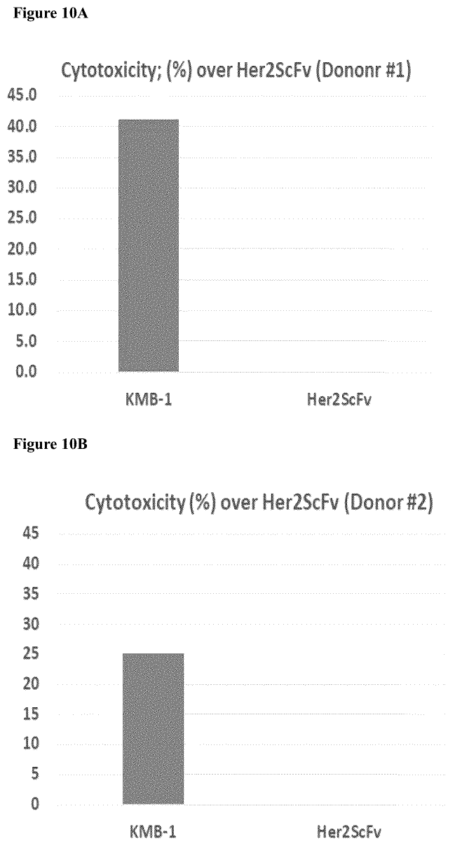

Next, the kinetics of the internalization process were analyzed in the Her2/Neu positive lines A431, BT474 and OvCar-3 following the protocol described in the Material and Methods section. FIGS. 9A-B, show a representative experiment performed on the A431 line. As a positivize control Herceptin (A) was used and demonstrated 42.37% intracellular presence at 24 hours and a maximal internalization of 54.4% at 48 hours post study initiation (FIG. 9A). As intracellular levels rose, the levels on the membrane correspondingly fell. The internalization kinetics of the Her2-ScFv-BCG-CMV chimera in the same study showed a similar trend with 43.8% intracellular expression at 24 hours but significantly higher internalization (71.3%) at 48 hours and even 72 hours (FIG. 9B). Internalization of BT474 demonstrated a similar trend with both Herceptin and Her2-ScFv-BCG-CMV (FIG. 9B).

### Functional Assays:

The final stage in Her2-ScFv-BCG-CMV's proof of concept analysis was to validate killing of target cells internalizing the chimera and expressing the SPD 24-mer BCG peptide. The effector cells in these studies were primary T-cells generated from naïve donors (as described in the Materials and Methods) against the synthetic 24-mer peptide. It is important to stress, that the same peptide is part of the antigenic payload in the Her2ScFv-BCG-CMV chimera. Her2-ScFv-BCG-CMV or Her2-ScFv-loaded autologous monocytes from the same donors used to generate the effectors were used as the target cells. As can be seen in FIGS. 10A-B, loading of targets cells with Her2-ScFv-BCG-CMV, but not Her2-ScFv lead to specific killing via autologous T-cells generated to the BCG 24-mer. This specific killing was observed in both donors. BCG 24-mer SPD-loaded monocytes were used as a positive control (data not shown). These results validated the antigen specific killing associated only with the Her2-ScFv-BCG-CMV chimera. In addition, it confirmed the ability of the 24-mer peptide to be cleaved from the Her2ScFv-BCG-CMV chimera inside target cells (in 2 different donors with different HLA repertoire), to reach the target cell's surface and enable BCG-24mer-specific T-cell killing.

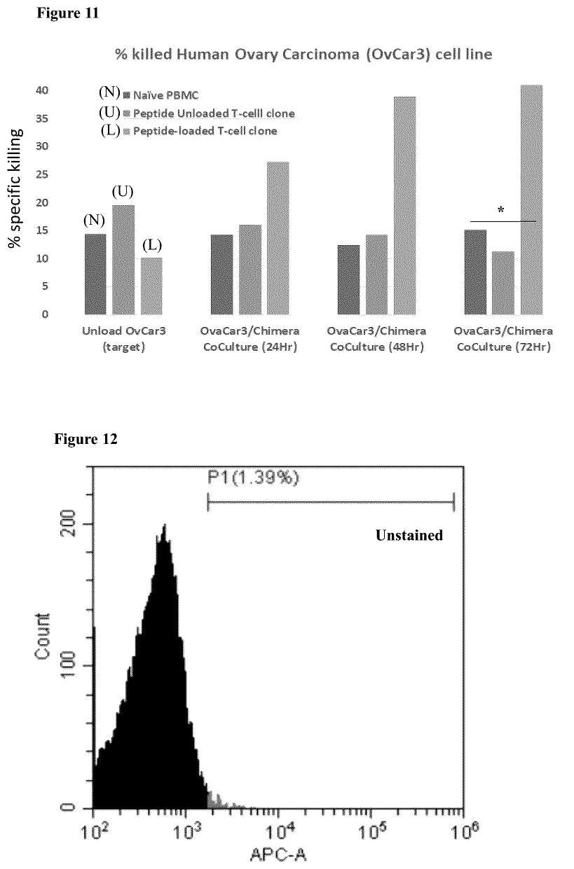

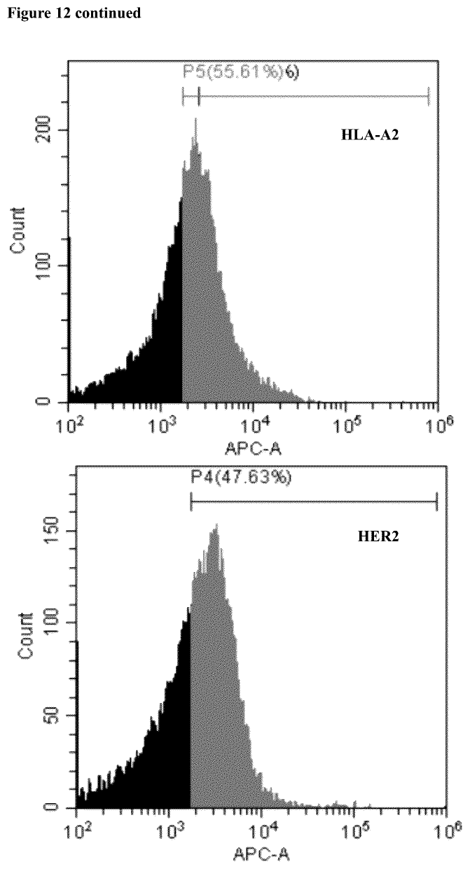

A follow up study used the same protocol to generate effector cells from naive HLA-A2.1-positive donors and Her2/Neu and HLA-A2.1 double positive OvCar-3 tumor cells as target cells. Based on the internalization kinetic properties of the chimera, the killing study was performed at the time range of 24-72 hours, in order to allow the chimera to efficiently internalized into the targets and reach the cell surface. Naïve T-cells and 24-mer unloaded T-cells were used as negative controls for non-specific killing. Similar to the results with donor monocytes, 24-mer BCG SPD-specific and HLA-A2.1 restricted killing of the OvCar-3 cell was observed exclusively with the 24-mer loaded T cells (FIG. 11). The tumor lysis was significantly higher than the non-specific killing with a peak at 48 and 72 hours of internalization. This result is significant as the tumor cells, unlike monocytes, do not phagocytose and so the uptake must be do to internalization after HER2 binding. Taken together these results demonstrate a potent chimera-mediated specific killing. This was observed in-spite of the fact that HLA-A2.1+/Her2Neu+ levels on OvCar-3 were around 50% each (FIG. 12); i.e., only about 20-40% of the cells were actually targets for killing.

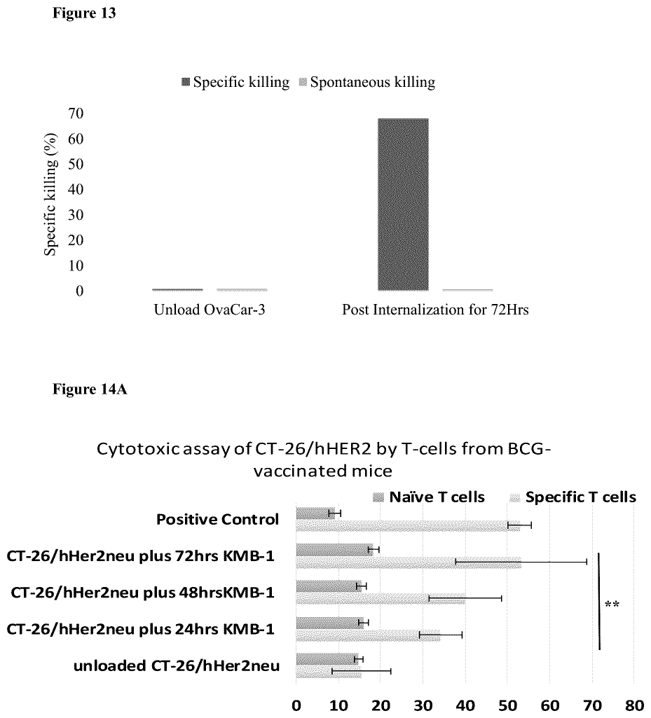

Finally, three more experiments were performed to further demonstrate the universality of the chimeric peptide. The first experiment was performed using commercially available anti-CMV 9-mer T-Cells from an HLA-A2.1 positive donor (Cellero, cat #1049). These T cells already target the 9-mer peptide that was part of chimeric molecule. HLA-A2.1 positive OvCar-3 tumor cells were used as before. Specific killing and spontaneous killing were calculated (Invitrogen LIVE/DEAD Cell-mediated cytotoxicity kit and Cytexpert software) for mock-loaded and Her2ScFv-BCG-CMV chimera loaded tumor cells. As expected, the 9-mer specific T cells induced a robust specific killing only of the chimera contacted tumor cell (FIG. 13). This demonstrates the universality of the chimera. Any T-cell epitope can be inserted downstream of the SP, so long as the subject has a pervious immunity against that epitope, or a suitable vaccine can be administered before/simultaneous to the administration of the chimera.

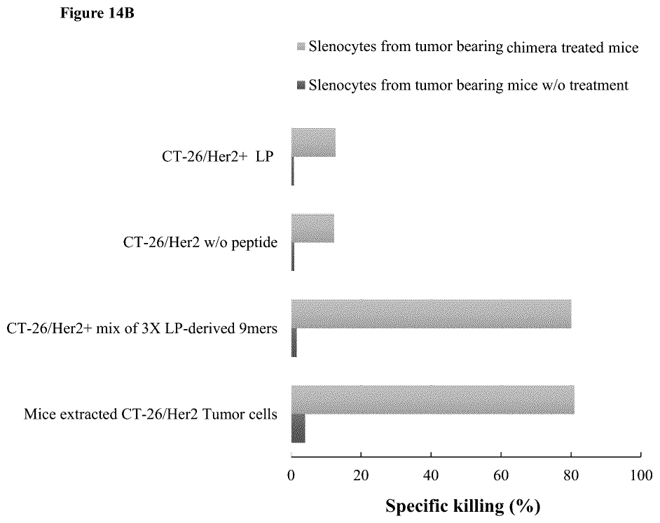

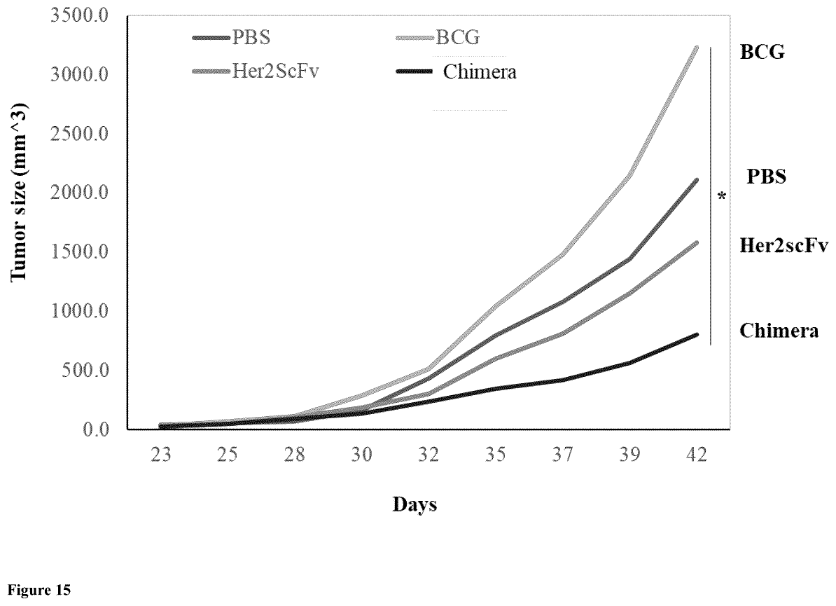

The second experiment demonstrated the effectiveness even of mouse immune cells after a single dose of the vaccine and with the known inferior binding of mouse MHC molecules to signal peptides as compared to humans. Three BALB/c mice were vaccinated with 5×10\hat{ }5 live BCG vaccine. Splenocytes were harvested from these mice 28 days later. CT-26/hHer2 (murine human Her2 positive transfected tumor cells) were contacted with the chimera and then specific killing was measured at various time points after the splenocytes were added. Naïve T cells not exposed to vaccine were used as control and the effector to target cell ratio for this experiment was 50:1. As can be seen in FIG. 14A, Her2ScFv-BCG-CMV loaded CT-26/hHer2 cells were indeed specifically killed by the murine splenocytes from mice that received the vaccine, whereas mock-loaded cells were not significantly killed. Further, naïve T cells did not show differential killing between the various cancer cells. This shows that even a single vaccine dose (without a booster) was sufficient to produce 24-mer specific splenocytes. It also validates the cross antigenic repertoires between the BCG vaccine and the 24-mer.

The final experiment was similar to the second, but in order to be more parallel to an in vivo situation, mice were injected with cells of the CT-26 mouse tumor cell line that express human HER2. Tumor bearing mice were treated with the chimera of the invention and 3 days after the final therapy the tumor was extracted. Splenocytes were extracted from the tumor bearing mice treated with the chimera or untreated tumor bearing mice. When extracted tumors were exposed to splenocytes from unvaccinated mice, very limited killing was observed (less than 15%, FIG. 14B). In contrast, when the tumors extracted from mice that received the chimera where mixed with the splenocytes, over 80% of cells were killed indicating the specificity and effectiveness of the chimera (FIG. 14B) and the activation phenotype of the effector T-cells. Additionally, CT-26 tumor cells not grown in mice, but exposed to 9-mers derived from the BCG 24-mer, were also killed only by the splenocytes from BCG vaccinated mice and at levels similar to the tumor cells (FIG. 14B). This demonstrates that it is the 9-mer epitopes that are displayed on the cell surface that are inducing the killing. The SP must be cleaved into roughly 9-mer fragments to be bound by the MHC molecules and shuttled to the cell surface.

Taken together, all of these results demonstrate that not only in the chimeric construct internalized, but both the first and second immunogenic peptides (BCG 24-mer and CMV 9-mer) are shuttled to the surface and are sufficient to induce specific killing by T cells in vitro.

### Example 6: In-Vivo Studies

In-vivo models were conducted to assess the immunogenic and efficacy of the construct of the invention in syngeneic mice.

Four groups of mice were tested. The first group (Group 1) was a control group that was injected with CT-26/hHer2 tumor cells, but otherwise was untreated. The second group (Group 2) was injected with the tumor transfected cells and also received the BCG vaccine. The third group (Group 3) was tumor injected, received the vaccine and also was treated with the control Her2scFv. The fourth group (Group 4) was tumor injected, received the vaccine and also was treated with the chimera of the invention. Tumor size was monitored as the measure of treatment effectiveness.

8 animals were included in each group and at Day 0 groups 2-4 all received a pre-treatment (priming) with the BCG vaccine. No booster with the vaccine was supplied as the previous experiment in which mice were vaccinated and splenocytes removed indicated that a single vaccination was sufficient to produce specific T cells. At Day 16, 1×10\hat{ }6 CT-26hHer2 cells were subcutaneously injected into all mice of all groups. Tumor size was measured from Day 23 and on until the conclusion of the study on Day 42. Starting on Day 28, Groups 3 and 4 received intratumor injections of 100 μg of therapeutic agent once every 3 days (based on the internalization kinetic). Group 3 received the Her2 scFv construct with no attached immunogenic peptides, while Group 4 received the Her2ScFv-BCG-CMV chimera, also 100 ug once every 3 days.

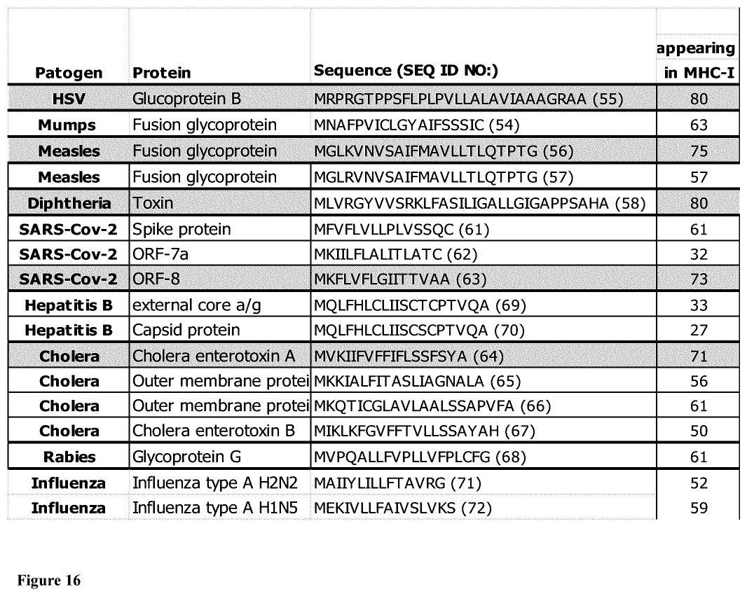

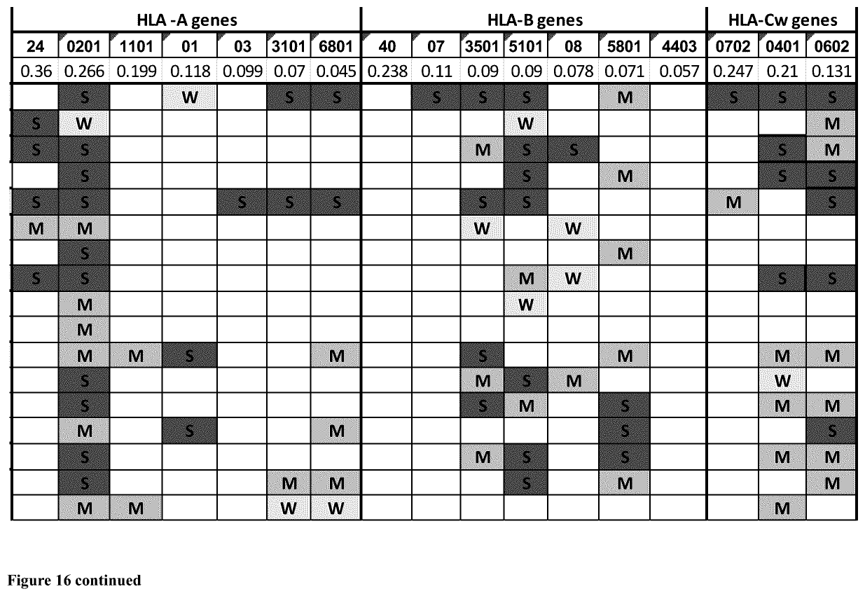

The results from these four groups of mice are summarized in Table 3 and FIG. 16 which provide T cell percentages in the tumor microenvironment in the 4 groups and the average tumor size for all mice of the group at the various time points shown, respectively. Control mice, that received the tumor cells and no other treatment, reached an average tumor size of 2000 mm\hat{ }3 on day 42 (Avr.=2113, STDVE=586). Surprisingly, mice that received the BCG vaccine before the tumor cell injection actually showed a worse outcome, with an average tumor size of over 3000 mm\hat{ }3 on day 42 (Avr.=3235, STDVE=1261). Treatment with the anti-Her2 scFv produced a modest improvement over the untreated group, with an average tumor size of about 1500 mm\hat{ }3 on day 42 (Avr.=1578, STDVE=529) which corresponds to a ˜25% decrease in tumor size. However, the chimera of the invention produced a far superior antitumor effect. At day 42 the average size of the tumor was only about 800 mm\hat{ }3 (Avr.=804, STDVE=433) which corresponds to a ˜62% reduction. The difference produced by the chimera in combination with the vaccine priming was statistically significant as compared to all the other test groups, demonstrated the surprising usefulness of the molecule. It bears noting that the Her2-targeting antibody-drug conjugate trastuzumab deruxtecan (DS-8201a) produced a comparable improvement over anti-Her2 antibody alone (see Iwata et al., 2018, “A Her2-targeting antibody-drug conjugate, trastuzumab deruxtecan (DS-8201a), enhances antitumor immunity in a mouse model”, Mol. Cancer Ther., July; 17(7):1494-1503, herein incorporated by reference in its entirety).

An investigation of the tumor resident T cells also produced surprising results. Firstly, before the first treatment the mice that received the vaccine priming showed a higher level of T cells in the tumor microenvironment as would be expected in animals that were immune stimulated. However, at the end of the treatment regimen very different numbers of T cells were observed in the tumor. On average 23,000 CD3 positive cells and of them 10000 CD3/CD44 positive cells were present in the fourth group that received the chimera. This was a robust and significant increase as compared to groups 2 and 3 which had only 2000 CD3 and of them 900 CD3/CD44 cells and 11000 CD3 and of them 3000 CD3/CD44 cells, respectively. Further, before the 1st treatment, 37.5% of the T cells were CD44/PD-1/LAG3 triple positive (indicating exhausted T cells) in all of groups 2-4. In contrast, at the end of treatment, exhausted T cells were nearly absent from the group 4 tumors but made up over 30% of T cells in groups 2 and 3. Taken together, this data clearly shows the in vivo effectiveness of the chimera.

TABLE 3
T cell distribution in the tumor microenvironment.

| | | | | | CD44/PD- |
|---|---|---|---|---|---|
| | CD3 | | CD44 | | 1/LAG3 |
| | % | | % | | % |
| | positive | Absolute No. @ | positive | Absolute No. @ | positive |
| Control group (Tumor) | Cells | 10 × 5 cells | Cells | 10 × 5 cells | Cells |
| Before the 1^{st} treatment | 24 | 24000 | 90 | 2200 | 11.3 |
| Post the 6^{th} treatment at | 4 | 4000 | 34 | 1400 | 14 |
| study completion | | | | | |
| | | | | | CD44/PD- |
| | CD3 | | CD3/CD44 | | 1/LAG3 |
| | % | | % | | % |
| Tumor & vaccine | positive | Absolute No. @ | positive | Absolute No. @ | positive |
| priming | Cells | 10 × 5 cells | Cells | 10 × 5 cells | Cells |
| Before the 1st treatment | 31.7 | 31700 | 78 | 25000 | 37.5 |
| Post the 6th treatment at | 2 | 2000 | 45 | 900 | 30.5 |
| study completion | | | | | |
| | | | | | CD44/PD- |
| | CD3 | | CD3/CD44 | | 1/LAG3 |
| Tumor & Vaccine | % | | % | | % |
| priming & Her2ScFv | positive | Absolute No. @ | positive | Absolute No. @ | positive |
| therapy | Cells | 10 × 5 cells | Cells | 10 × 5 cells | Cells |
| Before the 1st treatment | 31/7 | 31700 | 78 | 25000 | 37.5 |
| Post the 6th treatment at | 11 | 11000 | 25 | 3000 | 41.3 |
| study completion | | | | | |
| | | | | | CD44/PD- |
| | CD3 | | CD3/CD44 | | 1/LAG3 |
| Tumor & Vaccine | % | | % | | % |
| priming & KMB-1 | positive | Absolute No. @ | positive | Absolute No. @ | positive |
| therapy | Cells | 10 × 5 cells | Cells | 10 × 5 cells | Cells |
| Before the 1st treatment | 31.7 | 31700 | 78 | 25000 | 37.5 |
| Post the 6th treatment at | 23 | 23000 | 41 | 10000 | 1.1 |
| study completion | | | | | |

### Example 7: Testing Further Constructs

Alternative constructs containing a different targeting molecule (e.g., a different scFv) or a different immunogenic peptide (e.g., a different bacterial or viral sequence) are also tested. Constructs a designed as described hereinabove and tested in the in vitro and in vivo systems described. Cell lines/tumors are selected for expression of the tumor antigen targeted by the targeting molecule. Immunogenic peptides with known vaccines are selected for testing and the vaccine is used for priming of immune cells. Effectiveness of other chimeras is confirmed.

Target antigens and the sequences of their single chain binding sequences are selected from Table 1.

TABLE 1
cancer-proteins for targeting

| Target | Antibody | Antigen binding domain sequence |
|---|---|---|
| HER2 | trastuzumab | SEQ ID NO: 4 |
| | datopotamab | SEQ ID NO: 20 |
| Nectin 4 | enfortumab | SEQ ID NO: 23 |
| CD38 | belantamab | SEQ ID NO: 26 |
| TROP-2 | sacituzumab | SEQ ID NO: 29 |
| EGFR | disitamab | SEQ ID NO: 32 |
| CD19 | loncastuximab | SEQ ID NO: 35 |
| MSLN | anetumab | SEQ ID NO: 38 |
| CD25 | camidanlumab | SEQ ID NO: 41 |
| CEACAM5 | labetuzumab | SEQ ID NO: 44 |
| CD30 | brentuximab | SEQ ID NO: 79 |
| CD79B | polatuzumab | SEQ ID NO: 82 |
| FOLR1 | mirvetuximab | SEQ ID NO: 85 |
| BCMA | belantamab | SEQ ID NO: 88 |
| CD22 | inotuzumab | SEQ ID NO: 91 |
| B7-H3 | MGC-018 | SEQ ID NO: 94 |
| . . . | DS-7300 | SEQ ID NO: 97 |

Immunogenic peptides from the signal peptides of various non-human proteins are selected from those provided in Table 2. The MHC-I binding score for these peptides, as well as their HLA binding repertoire, is provided in FIG. 17.

TABLE 2
Immunogenic signal peptides

| | Available | | SPD sequence |
|---|---|---|---|
| Pathogen | Vaccine | Antigen | (SEQ ID NO:) |
| Tuberculosis | + | Antigen 85B | MTDVSRKIRAWGRRLMIGTAAAVVLPG |
| | | | LVGLAGGAATAGA (46) |
| | + | Lipoprotein lpqH | MKRGLTVAVAGAAILVAGLSGCSS (5) |
| | + | Uncharicterized | MLVLLVAVLVTAVYAFVHA (47) |
| | | protein | |
| | | Rv0476/MTO4941 | |
| | | prec | |
| | + | BPBP1 | MKIRLHTLLAVLTAAPLLLAAAGCGS |
| | | | (48) |
| | + | Hypothetical | MKTGTATTRRRLLAVLIALALPGAAVA |
| | | protein MT0213 | (49) |
| | + | Protease | MAAMWRRRPLSSALLSFGLLLGGLPLA |
| | | | APPLAGA (50) |
| | + | ATP dependent | MRFAQPSALSRFSALTRDWFTSTFAAPT |
| | | helicase putative | AAQA (51) |
| | + | Un char protein | MLLRKGTVYVLVIRADLVNAMVAHA |
| | | Rv1334/MT1376 | (52) |
| | | prec | |
| | + | Putative | MRPSRYAPLLCAMVLALAWLSAVAG |
| | | lipoprotein lpqV | (53) |
| | | prec | |
| Mumps | + | Fusion | MNAFPVICLGYAIFSSSICV (54) |
| | | glycoprotein | |
| HSV | − | Glucoprotein B | MRPRGTPPSFLPLPVLLALAVIAAAGRA |
| | | | A (55) |
| Measles | + | Fusion | MGLKVNVSAIFMAVLLTLQTPTGQ (56) |
| | | glycoprotein | |
| | + | Fusion | MGLRVNVSAIFMAVLLTLQTPTG (57) |
| | | glycoprotein | |
| Diphtheria | + | Toxin | MLVRGYVVSRKLFASILIGALLGIGAPPS |
| | | | AHA (58) |
| CMV | − | CMV En GlyP B | MESRIWCLVVCVNLCIVCLGAA (59) |
| | | 1-22 | |
| | − | CMV En GlyP H | MRPGLPPYLTVFTVYLLSHLPSQ (60) |
| | | 1-23 | |
| SARS-Cov- | + | Spike protein | MFVFLVLLPLVSSQC (61) |
| 2 | | | |
| | + | ORF-7a | MKIILFLALITLATC (62) |
| | + | ORF-8 | MKFLVFLGIITTVAA (63) |
| Cholera | + | Cholera | MVKIIFVFFIFLSSFSYA (64) |
| | | enterotoxin A | |
| | + | Outer membrane | MKKIALFITASLIAGNALA (65) |
| | | protein V | |
| | + | Outer membrane | MKQTICGLAVLAALSSAPVFA (66) |
| | | protein W | |
| | + | Cholera | MIKLKFGVFFTVLLSSAYAH (67) |
| | | enterotoxin B | |
| Rabies | + | Glycoprotein G | MVPQALLFVPLLVFPLCFG (68) |
| Hepatitis B | + | external core | MQLFHLCLIISCTCPTVQA (69) |
| | | antigen | |
| | + | Capsid protein | MQLFHLCLIISCSCPTVQA (70) |
| Influenza | + | Hemagglutinin | MAIIYLILLFTAVRG (71) |
| type A | | | |
| H2N2 | | | |
| Influenza | + | Hemagglutinin | MEKIVLLFAIVSLVKS (72) |
| type A | | | |
| H1N5 | | | |
| Influenza A | + | | MNTQILVFALVAIIPTNA (73) |
| virus | | | |

Various cleavable peptides and linkers (both flexible and rigid) are also tested.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A chimeric polypeptide comprising:
a. a first subunit comprising an antigen-binding domain of a cancer surface antigen;
b. a second subunit comprising at least one immunogenic peptide comprising a signal peptide from a non-human protein or a fragment thereof capable of binding by a major histocompatibility complex (MHC) molecule; and
c. a third subunit comprising a cleavable moiety, wherein said cleavable moiety is cleaved in an endosome or lysosome of a human;
wherein said third subunit is between said first and second subunits in said polypeptide.

2. The chimeric polypeptide of claim 1, wherein said first subunit comprises a single chain antibody (scFv), a single domain antibody (sdAb) or an scFv linked to an Fc region.

3. (canceled)

4. (canceled)

5. The chimeric polypeptide of claim 1, wherein said cancer surface antigen is a cancer specific surface antigen which is not expressed on non-cancer cells or is significantly lower expressed on non-cancer cells than on cancer cells and is selected from the group consisting of: receptor tyrosine-protein kinase ERBB2 (HER2), CD30, CD79B, Nectin 4 (NECTIN4), CD38, CD22, tumor-associated calcium signaling transducer 2 (TROP-2), epidermal growth factor receptor (EGFR), CD19, Folate Receptor alpha (FOLR1), mesothelin (MSLN), CD25, B-cell maturation antigen (BCMA), CD276, and carcinoembryonic antigen-related cell adhesion molecule 5 (CEACAM5).

6. The chimeric polypeptide of claim 5, wherein:
a. said cancer antigen is HER2 and said antigen binding domain comprises SEQ ID NO:18 and SEQ ID NO: 19;
b. said cancer antigen is Nectin-4 and said antigen binding domain comprises SEQ ID NO:21 and SEQ ID NO: 22;
c. said cancer antigen is CD38 and said antigen binding domain comprises SEQ ID NO:24 and SEQ ID NO: 25;
d. said cancer antigen is TROP-2 and said antigen binding domain comprises SEQ ID NO:27 and SEQ ID NO: 28;
e. said cancer antigen is EGFR and said antigen binding domain comprises SEQ ID NO:30 and SEQ ID NO: 31;
f. said cancer antigen is CD19 and said antigen binding domain comprises SEQ ID NO:33 and SEQ ID NO: 34;
g. said cancer antigen is MSLN and said antigen binding domain comprises SEQ ID NO:36 and SEQ ID NO: 37;
h. said cancer antigen is CD25 and said antigen binding domain comprises SEQ ID NO:39 and SEQ ID NO: 40;
i. said cancer antigen is CEACM5 and said antigen binding domain comprises SEQ ID NO:42 and SEQ ID NO: 43;
j. said cancer antigen is CD30 and said antigen binding domain comprises SEQ ID NO:77 and SEQ ID NO: 78;
k. said cancer antigen is CD79B and said antigen binding domain comprises SEQ ID NO:80 and SEQ ID NO: 81;
l. said cancer antigen is FLOLR1 and said antigen binding domain comprises SEQ ID NO:83 and SEQ ID NO: 84;
m. said cancer antigen is BCMA and said antigen binding domain comprises SEQ ID NO:86 and SEQ ID NO: 87;
n. said cancer antigen is CD22 and said antigen binding domain comprises SEQ ID NO:89 and SEQ ID NO: 90;
o. said cancer antigen is CD276 and said antigen binding domain comprises SEQ ID NO:92 and SEQ ID NO: 93;
p. said cancer antigen is CD276 and said antigen binding domain comprises SEQ ID NO:95 and SEQ ID NO: 96; or
q. said cancer antigen is HER2 and said antigen binding domain comprises SEQ ID NO: 1 and SEQ ID NO: 2 separated by an amino acid linker or SEQ ID NO: 4.

7. (canceled)

8. (canceled)

9. (canceled)

10. The chimeric polypeptide of claim 1, wherein said at least one immunogenic peptide comprises a sequence from a vaccine suitable for administration to humans or comprises a sequence from a pathogen to which humans have natural immunity.

11. (canceled)

12. The chimeric polypeptide claim 1, wherein said non-human protein is selected from a viral protein, a bacterial protein and a parasite protein, optionally said non-human protein is selected from a tuberculosis protein, a mumps protein, a herpes simplex virus (HSV) protein, a measles protein, a diphtheria protein, a cytomegalovirus (CMV) protein, a severe acute respiratory syndrome coronavirus 2 (SARS-CoV-2) protein, a cholera protein, a rabies protein, a hepatitis B protein, and an influenza type A protein or is selected from a protein provided in Table 2.

13. (canceled)

14. (canceled)

15. The chimeric polypeptide of claim 12, wherein said second subunit comprises a sequence selected from SEQ ID NO: 5 and 46-73.

16. (canceled)

17. The chimeric polypeptide of claim 1, wherein said cleavable moiety is a furin-cleavable linker comprises a sequence selected from RXBR, wherein X is any amino acid and B is a positively charged amino acid selected from R and K (SEQ ID NO: 45) and SEQ ID NO: 6.

18. (canceled)

19. (canceled)

20. The chimeric polypeptide of claim 1, wherein said second subunit further comprises a second immunogenic peptide from a non-human protein and wherein said second immunogenic peptide is C-terminal to said immunogenic peptide comprising a signal peptide or a fragment thereof.

21. The chimeric polypeptide of claim 20, wherein at least one of:
a. said second immunogenic peptide comprises a sequence from a vaccine suitable for administration to humans;
b. said second immunogenic peptide comprises a sequence to which humans have a natural immunity; and
C. said second immunogenic peptide consists of SEQ ID NO: 11.

22. (canceled)

23. (canceled)

24. The chimeric polypeptide of claim 21, wherein
said second subunit consists of the sequence MKRGLTVAVAGAAILVAGLSGCSS GGSGGSGGSNLVPMVATV (SEQ ID NO: 74).

25. The chimeric polypeptide of claim 1, wherein any one of said first subunit, said second subunit and said third subunit are separated by a linker, or wherein said chimeric polypeptide further comprises an N-terminal leader peptide, a C-terminal affinity tag or both, optionally wherein said linker is a flexible glycine and serine linker.

26. (canceled)

27. (canceled)

28. The chimeric polypeptide of claim 1, comprising an amino acid sequence selected from SEQ ID NO: 12-16, consisting of an amino acid sequence selected from SEQ ID NO: 14 to 16 or comprising at least 90% homology to an amino acid sequence selected from SEQ ID NO: 14 to 16 and being capable of binding said cancer surface antigen on a surface of a target cell and express said immunogenic peptide on a surface of said target cell.

29. (canceled)

30. A nucleic acid molecule comprising a nucleotide sequence encoding a chimeric polypeptide of claim 1, and optionally further comprising at least one regulatory element operatively linked to said nucleotide sequence and capable of driving expression of said nucleotide sequence in a target cell.

31. (canceled)

32. A host cell comprising a nucleic acid molecule of claim 30.

33. A pharmaceutical composition comprising a chimeric polypeptide of claim 1 and a pharmaceutically acceptable carrier, excipient or adjuvant, optionally wherein said pharmaceutical composition is configured for systemic administration.

34. (canceled)

35. A method of expressing an immunogenic peptide on a surface of a target cell expressing said cancer surface antigen, the method comprising contacting said target cell with a pharmaceutical composition of claim 33, thereby expressing said immunogenic peptide on a surface of said target cell.

36. A method of preventing, ameliorating, or treating a cancer expressing said cancer surface antigen in a subject in need thereof, the method comprising administrating to said subject a pharmaceutical composition of claim 33, thereby preventing, ameliorating, or treating a cancer.

37. The method of claim 36, wherein said subject has already been vaccinated with a vaccine comprising an amino acid sequence present in said second subunit or wherein said method further comprises at least one of:
(a) administering a vaccine to said subject before administering said chimeric polypeptide or pharmaceutical composition, wherein said vaccine comprises an amino acid sequence present in said second subunit; and
(b) treating said subject with adoptive T cell transfer or chimeric antigen receptor (CAR) therapy wherein said T cell or CAR is specific to a sequence present in said second subunit.

38. (canceled)

39. The method of claim 37, wherein said vaccine is the Bacillus Calmette-Guerin (BCG) anti-tuberculosis vaccine and said second subunit comprises an amino acid sequence selected from SEQ ID NO: 5 and 46-53.

40. (canceled)

41. (canceled)
